SEQUENCE LISTING (12 sequences)

SEQ ID NO: 1 - DNA, 957 bp, Bacillus licheniformis
atgctacgatcgcagcgaacgaagaaaaagagactaagaaaatgggtgaaatactcactgtttttcattgctttaatcctgacggcgacggcagccgcaggcggctatgcgtactataaagtggcgaacgcctccaaagatgcgcaagtcaccctttccagaggtgaacagtccgtcaagcggattaaagagttcaaccctaaaaaagataacttctccatcctgttgatggggattgatgcccgtcccggacaaagcatggacaaagaacgaagcgatgcgatggttcttgccacgttcaacagaaaagacaaatcggtgaaactattgagcatcccgcgggattcctatgtcaacatccccggacgcggctacgataaaataacgcatgcccactcattaggcgggcgcgaccttagtacagaaaccgtcgaaaacctgctcgacattccggttgattacgtcatggaagccaatttttcagctttcaaggaagtagttgatgaactcggcggtgtaccgatcacgatcaaagaagactatattgtccggcagattaaaaaggatacgaaaggcaaagtcaaccttcaaacaggcgagcagacgctgggcggagaagaagccctcgcttatgtaagaacgagaaaagccgatacggacttgaagcgcggcgagcgccagatggaagtcatcaaatcgattatcaacaaatccaaatcgcttacttccattccggcttacgacgatatccttgatacagtcggaaagaacgtatcgatgaacttgtcgctgaatgatgcaatcggactgatcccgtttatgacgtccattcaaacggtcgatacccttcaattgaaagggagcgattatcaaccgggcaaagtctactatttccagcttgatcaggaaaacctcaacgaaattaagcaggaattaaaacagcagttagatgtataa

SEQ ID NO: 2 - DNA, 861 bp, Bacillus licheniformis
atgagattaattgcgatcgatttagacgggactcttttaaataccgaaagcaaaatatccgcagaaaaccgggccgctatcaaaaaggcgctcatttcagactgcatcgtaaccatttgcacgggacgggccacatttgacgtaaaagcgcttctgaacgatttggatattccgattatcgctgcaaacgggggaacggtccacgatttcaactataaactcctaagcagaacgctaatggacaaggaagcgggaaaaaaggccgcccaatacttaacagatcatcaaatctattttgaggtttatacggacgatgcgctgctttctccttttgacggagaacagaaacttaaagcagagctcgatatcgtcacaagcgccaaccctgatgaagacagggatacgctttggcaaggcgcactgactcagtttaaacaattcggcatcaaacccgtccccgacattcgcgagatttttaacggagacgaacatatttataagctgttgtgcttctcatttgatatggaaaagctgaagaaagcaaaagatgatctttctgcctttccaaaactgggactcacttcttccggaagacatatcatcgagattctgcctgatgaatccggcaaaggccgcgctttaaaagagctggcccgccactatggaatcgagaaaaaagacatctacgcgatcggcgacagcccgaacgatctgtcaatgtttgaggaagccggccatcggatcgcgatgggcaatgcaatcgatgagctaaagcatatcagcacctttatcacgaaaagcaatgacgaaaacggcgtcgcttatttcattaacaaactgctggaaaatgaatatcaataa

SEQ ID NO: 3 - DNA, 1245 bp, Bacillus licheniformis
ttgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttgggagcagtgtcggccgtcagcagctcgaaaagggggaccttgtctttttcaagaatgccgagctggaatcggacggaccgacccatgtcgccatctatttgggaaatgatcaaatcatccacagcacaaaatcaaacggggttgtcgtgacaaagcttgaaggcagctcttactggagctcggggtattttaaagcgaaaaggatcacaaaagagcctgagatttcgatggatcctgtcgttcaaaaagcaaaaagctatgtcggtgttccttatgtatttggaggcaactctccggatctcggatttgactgttcggggttgacccaatacgtcttcagagaggtgctcggcgtttatttgccaaggtcggctgaacagcaatgggctgtcggtcaaaaggtgaagcttgaagatatccggccgggtgatgttttgtttttcagcaatacgtacaaaccgggaatatcccataacggcatctatgccgggggcgggcggtttatccatgcgagccgttcaaataaagtgacgatatcctacttgtcggcttcctattggcagaagaagttcacaggagtcagacgttttgacaacatgtccctgccaaaaaatccgattgtatccgaagccatcaggcatatcggcgaagtcggttatcaaaaaggcggcacatcgcctaaagaaggctttgatacggctgggtttatccaatatgtctacaaaacggcggcaggagtggagcttccgaggtatgctgacaaacaatacagcacgggtaagaaaattaccaaacaggagcttgagcctggagacatcgtcttctttaaaggaaccactgttatgaatcccgccatctatatcggaaacggccaggtcgttcttgtcaccttgtctgccggtgtaacgacagcagatatggagacgagcgcctattggaaagataaatacgccggaagcgtcagaattgagtag

SEQ ID NO: 4 - DNA, 1170 bp, Bacillus licheniformis
atgaaaaaacaactgaactttcaggaaaaactgctgaagttgacgaagcaggagaaaaagaaaacaaacaagcacgtctttatcgtattgcccgttattttctgtttaatgtttgtctttacttgggtcggaagcgccaaaactccttcgcaaatggacaaaaaagaagatgccaagcttacagctacttttgttggcgatatcatgatgggaagaaacgtagaaaaagtgacaaacttgcacggttcggaaagtgtcttcaaaaatgtgaagccgtactttaatgtgtcagattttatcacaggaaactttgaaaaccctgtaaccaatgcaaaggactatcaagaggcagaaaagaacatccatctgcaaacgaatcaagaatcagtcgaaacattgaaaaagctgaacttcagcgtactgaattttgccaacaaccatgcgatggactacggggaagacggtttgaaggatacgctcaataaattttcaaatgagaatctggagcttgtcggagcaggaaataatcttgaagacgcgaaacagcacgtatcctatcagaatgtgaacggcgtaaaaattgcaacgctcggttttacagacgtctacacaaagaactttacagccaaaaagaacagaggcggagtgctgccgctcagtccgaaaatctttattccaatgattgcggaagcatcgaaaaaagcggatcttgtccttgtccatgtgcactggggacaagaatatgacaatgaaccgaacgacagacagaaggatctggccaaggcgattgcagatgccggagcagatgtcatcatcggcgctcatccccatgttctcgaaccgatcgaagtgtataacggtactgtgattttctacagcctcggcaactttgtatttgatcagggctggtcaagaacacgggacagcgcgcttgtacaataccatttaatgaatgacggcaaagggcgctttgaggtaacgcctctcaacattcgcgaagcaacgccgacgcctttaggcaagagcgacttcttaaaacgaaaagcgatcttccgtcaattgacaaaaggaacaaacctcgactggaaagaagagaacggaaaattaacgtttgaagtcgatcatgcggacaagctgaaaaataataaaaacggagtggtgaacaaatga

SEQ ID NO: 5 - DNA, 450 bp, Bacillus licheniformis
atgtttggatcagatttatatatcgccctcattttaggagtcttactcagtttgatttttgcagagaaaacgggaattgtaccagccggcctcgtcgtaccgggttatttgggacttgtcttcaatcagccgattttcatgctgctcgttctttttgtcagtttgctgacgtatgtcatcgtgaaattcggactttccaaaattatgattctatacggacgcagaaaattcgcagcaatgctgattacgggaattcttttgaaaatcggttttgattttatatatccggtgatgccgtttgagattgccgaattcaggggaatcggaatcatcgtgccggggctgatcgccaataccattcaaagacagggattaacgattacgcttggaagtacgcttttattgagcggagcaacattcgtcattatgtatgcttactatctaatctaa

SEQ ID NO: 6 - DNA, 1182 bp, Bacillus licheniformis
atgtgggtaatgctattagcctgtgtgatcgttgttgggatcggcatttatgaaaaaaggcgccaccagcaaaatatcgatgcgctgcctgtccgagtgaacatcaacggtatacgcggaaagtccacggtgacaagattaacaacagggatattaatcgaagcaggctacaaaacagtaggaaaaacaaccgggacagacgcaaggatgatttattgggacacaccggaagagaagccgatcaaaagaaagccgcaagggccgaatatcggagagcagaaggaggttatgaaagaaacggtggaaagaggggccaatgcgattgtcagtgagtgcatggccgttaatcctgattaccaaatcatctttcaggaagaattgcttcaggctaatatcggcgtgatcgtgaacgtgctggaggatcacatggatgtgatgggaccgactttggatgaaatcgcagaagcattcacagcaaccattccttataatggacatttggttattactgatagtgagtataccgatttctttaagcaaattgcaaaagaaaggaacacaaaagtcatcgtcgcagacaattctaaaataacagatgaatacctcagacagtttgagtacatggtattccctgataatgcgtctcttgcgctcggtgtagctcaagcgttgggcattgacgaagaaaccgcctttaaaggcatgctgaatgcgccgcctgatccgggagccatgagaattctgccgctgatgaacgccaagaatcccggacatttcgtcaacggttttgcggccaatgacgcagcttccactttaaacatttggaagcgtgtaaaagaaataggctatcctacggatcagccgatcgtcattatgaactgccgcgccgacagggtagacagaacacagcagtttgcggaagatgtccttccttatattgaagcaagtgaacttgtgctgattggagaaacaacagagccgatcgtcaaagcatatgaagcaggcaaaattcctgcggacaagctgtttgattttgagcacaaatcaacggaagaaatcatgttcatgctgaaaaacaagcttgagggccgcgttatttacggagtcggaaatatccacggagcagcggagcctctcattgaaaaaatacaagattacaagattaagcagctcgttagctag

SEQ ID NO: 7 - protein, 318 aa, Bacillus licheniformis
MLRSQRTKKKRLRKWVKYSLFFIALILTATAAAGGYAYYKVANASKDAQVTLSRGEQSVKRIKEFNPKKDNFSILLMGIDARPGQSMDKERSDAMVLATFNRKDKSVKLLSIPRDSYVNIPGRGYDKITHAHSLGGRDLSTETVENLLDIPVDYVMEANFSAFKEVVDELGGVPITIKEDYIVRQIKKDTKGKVNLQTGEQTLGGEEALAYVRTRKADTDLKRGERQMEVIKSIINKSKSLTSIPAYDDILDTVGKNVSMNLSLNDAIGLIPFMTSIQTVDTLQLKGSDYQPGKVYYFQLDQENLNEIKQELKQQLDV

SEQ ID NO: 8 - protein, 286 aa, Bacillus licheniformis
MRLIAIDLDGTLLNTESKISAENRAAIKKALISDCIVTICTGRATFDVKALLNDLDIPIIAANGGTVHDFNYKLLSRTLMDKEAGKKAAQYLTDHQIYFEVYTDDALLSPFDGEQKLKAELDIVTSANPDEDRDTLWQGALTQFKQFGIKPVPDIREIFNGDEHIYKLLCFSFDMEKLKKAKDDLSAFPKLGLTSSGRHIIEILPDESGKGRALKELARHYGIEKKDIYAIGDSPNDLSMFEEAGHRIAMGNAIDELKHISTFITKSNDENGVAYFINKLLENEYQ

SEQ ID NO: 9 - protein, 414 aa, Bacillus licheniformis
LIKKAANKKLVLFCGIAVLWMSLFLTNHNDVRADTIGEKIAETARQLEGAKYSYGGEKPKTGFDSSGFVQYVFQSLDITLPRTVKEQSTLGSSVGRQQLEKGDLVFFKNAELESDGPTHVAIYLGNDQIIHSTKSNGVVVTKLEGSSYWSSGYFKAKRITKEPEISMDPVVQKAKSYVGVPYVFGGNSPDLGFDCSGLTQYVFREVLGVYLPRSAEQQWAVGQKVKLEDIRPGDVLFFSNTYKPGISHNGIYAGGGRFIHASRSNKVTISYLSASYWQKKFTGVRRFDNMSLPKNPIVSEAIRHIGEVGYQKGGTSPKEGFDTAGFIQYVYKTAAGVELPRYADKQYSTGKKITKQELEPGDIVFFKGTTVMNPAIYIGNGQVVLVTLSAGVTTADMETSAYWKDKYAGSVRIE

SEQ ID NO: 10 - protein, 389 aa, Bacillus licheniformis
MKKQLNFQEKLLKLTKQEKKKTNKHVFIVLPVIFCLMFVFTWVGSAKTPSQMDKKEDAKLTATFVGDIMMGRNVEKVTNLHGSESVFKNVKPYFNVSDFITGNFENPVTNAKDYQEAEKNIHLQTNQESVETLKKLNFSVLNFANNHAMDYGEDGLKDTLNKFSNENLELVGAGNNLEDAKQHVSYQNVNGVKIATLGFTDVYTKNFTAKKNRGGVLPLSPKIFIPMIAEASKKADLVLVHVHWGQEYDNEPNDRQKDLAKAIADAGADVIIGAHPHVLEPIEVYNGTVIFYSLGNFVFDQGWSRTRDSALVQYHLMNDGKGRFEVTPLNIREATPTPLGKSDFLKRKAIFRQLTKGTNLDWKEENGKLTFEVDHADKLKNNKNGVVNK

SEQ ID NO: 11 - protein, 149 aa, Bacillus licheniformis
MFGSDLYIALILGVLLSLIFAEKTGIVPAGLVVPGYLGLVFNQPIFMLLVLFVSLLTYVIVKFGLSKIMILYGRRKFAAMLITGILLKIGFDFIYPVMPFEIAEFRGIGIIVPGLIANTIQRQGLTITLGSTLLLSGATFVIMYAYYLI

SEQ ID NO: 12 - protein, 393 aa, Bacillus licheniformis
MWVMLLACVIVVGIGIYEKRRHQQNIDALPVRVNINGIRGKSTVTRLTTGILIEAGYKTVGKTTGTDARMIYWDTPEEKPIKRKPQGPNIGEQKEVMKETVERGANAIVSECMAVNPDYQIIFQEELLQANIGVIVNVLEDHMDVMGPTLDEIAEAFTATIPYNGHLVITDSEYTDFFKQIAKERNTKVIVADNSKITDEYLRQFEYMVFPDNASLALGVAQALGIDEETAFKGMLNAPPDPGAMRILPLMNAKNPGHFVNGFAANDAASTLNIWKRVKEIGYPTDQPIVIMNCRADRVDRTQQFAEDVLPYIEASELVLIGETTEPIVKAYEAGKIPADKLFDFEHKSTEEIMFMLKNKLEGRVIYGVGNIHGAAEPLIEKIQDYKIKQLVS

(19) United States
(12) Patent Application Publication
(10) Pub. No.: US 2016/0002591 A1
(43) Pub. Date: Jan. 7, 2016

(54) Inactivation of Glutamyl Polypeptide Synthesis in Bacillus

(71) Applicant: NOVOZYMES INC., Davis, CA (US)

(72) Inventors: Randy Berka, Davis, CA (US); Michael Rey, Davis, CA (US); Preethi Ramaiya, Sunnyvale, CA (US)

(73) Assignee: NOVOZYMES INC., Davis, CA (US)

(21) Appl. No.: 14/803,808

(22) Filed: Jul. 20, 2015

Related U.S. Application Data
(62) Division of application No. 13/961,146, filed on Aug. 7, 2013, now abandoned.
Application No. 13/961,146 is a division of application No. 12/516,426, filed on Jun. 11, 2009, now abandoned.
(60) Provisional application No. 60/861,992, filed on Nov. 29, 2006.

(51) Int. Cl. C12N 1/20; C12R 1/10

(52) U.S. Cl. CPC C12N 1/20; C12R 1/10

(57) ABSTRACT

The present invention relates to isolated polynucleotides of the chromosome of Bacillus licheniformis SJ1904 that encode biologically active substances and to nucleic acid constructs, vectors, and host cells comprising the polynucleotides as well as methods for producing biologically active substances encoded by the polynucleotides and to methods of using the isolated polynucleotides of the complete chromosome of Bacillus licheniformis SJ1904.

### CROSS-REFERENCE TO RELATED APPLICATIONS

This application is a divisional application of U.S. application Ser. No. 13/961,146 filed on Aug. 7, 2013, now pending, which is a divisional application of U.S. application Ser. No. 12/516,426 filed on Jun. 11, 2009, now abandoned, which is a 35 U.S.C. 371 national application of PCT/US2007/024746 filed on Nov. 29, 2007, which claims priority or the benefit of U.S. Provisional Application No. 60/861,992 filed on Nov. 29, 2006.

### REFERENCE TO SEQUENCE LISTING

This application contains a Sequence Listing in computer readable form, which is incorporated herein by reference.

### REFERENCE TO A DEPOSIT OF BIOLOGICAL MATERIAL

This application contains a reference to a deposit of biological material, which deposit is incorporated herein by reference.

### BACKGROUND OF THE INVENTION

1. Field of the Invention

The present invention relates to features (polynucleotides) of the complete chromosomal DNA molecule of Bacillus licheniformis SJ1904 that encode biologically active substances and to nucleic acid constructs, vectors, and host cells comprising the features. The present invention also relates to methods for producing biologically active substances encoded by the features and to methods of using the isolated features derived from the complete chromosomal DNA molecule of Bacillus licheniformis SJ1904.

2. Description of the Related Art

Microbes have evolved for some 3.8 billion years and make up most of the earth's biomass. They are found in virtually every environment, surviving and thriving in extremes of heat, cold, radiation, pressure, salt, acidity, and darkness. Often in these environments, no other forms of life are found and the only nutrients come from inorganic matter. The diversity and range of their environmental adaptations indicate that microbes long ago “solved” many problems for which scientists are still actively seeking solutions. The value in determining the complete genome sequence of microbes is that it provides a detailed blueprint for the organism revealing biochemical pathways, substrates, intermediates, and end products as well as regulatory networks, and evolutionary relationships to other microbes. A complete manifest of proteins, both structural and catalytic, is encoded as a list of features in the DNA molecule comprising the genome, as well as their likely cellular location.

Knowledge about the enormous range of microbial capacities has broad and far-reaching implications for environmental, energy, health, and industrial applications, such as cleanup of toxic-waste, production of novel therapeutic and preventive agents (drugs and vaccines), energy generation and development of renewable energy sources, production of chemical catalysts, reagents, and enzymes to improve efficiency of industrial processes, management of environmental carbon, nitrogen and nutrient cycling, detection of disease-causing organisms, monitoring of the safety of food and water supplies, use of genetically altered bacteria as living sensors (biosensors) to detect harmful chemicals in soil, air, or water, and understanding of specialized systems used by microbial cells to live in natural environments.

Bacillus licheniformis is a gram positive spore-forming bacterium that is widely distributed as a saprophytic organism in the environment. Unlike most other bacilli that are predominantly aerobic, Bacillus licheniformis is a facultative anaerobe that may allow it to grow in additional ecological niches. This species produces a diverse assortment of extracellular enzymes that are believed to contribute to the process of nutrient cycling in nature (Claus, D. and Berkeley, R. C. W., 1986, In Bergey's Manual of Systematic Bacteriology, Vol. 2., eds. Sneath, P. H. A. et al., Williams and Wilkins Co., Baltimore, Md., pp. 1105-1139). Certain Bacillus licheniformis isolates are capable of denitrification but, the relevance of this characteristic to environmental denitrification may be small since the species generally persists in soil as endospores (Alexander, M., 1977, Introduction to Soil Microbiology. John Wiley and Sons, Inc., New York).

There are numerous industrial and agricultural uses for Bacillus licheniformis and its extracellular products. The species has been used for decades in the manufacture of industrial enzymes including several proteases, alpha-amylase, penicillinase, pentosanase, cycloglucosyltransferase, beta-mannanase, and several pectinolytic enzymes, owing largely to its ability to secrete sizeable amounts of degradative enzymes. Bacillus licheniformis is also used to produce peptide antibiotics such as bacitracin and proticin, in addition to a number of specialty chemicals such as citric acid, inosine, inosinic acid, and poly-gamma-glutamic acid. The proteases from Bacillus licheniformis are used in the detergent industry as well as for dehairing and batting of leather (Eveleigh, D. E., 1981, Scientific American 245, 155-178). Amylases from Bacillus licheniformis are deployed for the hydrolysis of starch, desizing of textiles, and sizing of paper (Erickson, R. J., 1976, In Microbiology, ed. Schlesinger, D. (Am. Soc. Microbiol., Washington, D.C.), pp. 406-419.). Certain strains of Bacillus licheniformis have shown efficacy to destroy fungal pathogens affecting maize, grasses, and vegetable crops (U.S. Pat. No. 5,589,381; U.S. Pat. No. 5,665,354). As an endospore-forming bacterium, the ability of the organism to survive under unfavorable environmental conditions may enhance its potential as a natural control agent.

Bacillus licheniformis can be differentiated from other bacilli on the basis of metabolic and physiological tests (Logan, N. A. and Berkeley, R. C. W., 1981, In The Aerobic Endospore-Forming Bacteria: Classification and Identification, eds. Berkeley, R. C. W. and Goodfellow, M., Academic Press, Inc., London, pp. 106-140; O'Donnell, A. G., Norris, J. R., Berkeley, R. C. W., Claus, D., Kanero, T., Logan, N. A., and Nozaki, R., 1980, Internat. J. Systematic Bacteriol. 30: 448-459). However, biochemical and phenotypic characteristics may be ambiguous among closely related species. Lapidus et al. (Lapidus, A., Galleron, N., Andersen, J. T., Jørgensen, P. L. Ehrlich, S. D., and Sorokin, A., 2002, FEMS Microbiol. Lett. 209: 23-30) constructed a physical map of the Bacillus licheniformis strain ATCC 14580 chromosome using a PCR approach, and established a number of regions of co-linearity where gene content and organization were conserved with the Bacillus subtilis chromosome. In addition, Rey et al. (Rey, M. W, Ramaiya, P, Nelson, B. A., Brody-Karpin, S. D., Zaretsky, E. J., Tang, M., Lopez de Leon, A., Xiang, H., Gusti, V., Clausen, I. G., Olsen, P. B., Rasmussen, M. D., Andersen, J. T., Jørgensen, P. L., Larsen, T. S., Sorokin, A., Bolotin, A., Lapidus, A., Galleron, N., Ehrlich, S. D., and Berka, R. M. 2004. Complete genome sequence of the industrial bacterium Bacillus licheniformis and comparisons with closely related Bacillus species. Genome Biol. 5: R77) published the complete genome sequence of the Bacillus licheniformis type strain ATCC 14580. They determined that the genome of Bacillus licheniformis ATCC 14580 comprises a circular chromosome of 4,222,336 base-pairs (bp) containing 4,208 predicted protein-coding genes with an average size of 873 bp, seven rRNA operons, and 72 tRNA genes. The Bacillus licheniformis ATCC 14580 chromosome contains large regions that are colinear with the genomes of Bacillus subtilis strain 168 and Bacillus halodurans strain C-125, and approximately 80% of the predicted Bacillus licheniformis ATCC 14580 coding sequences have Bacillus subtilis orthologs. However, despite the unmistakable organizational similarities between these Bacillus licheniformis and Bacillus subtilis genomes, there are notable differences in the numbers and locations of prophages, transposable elements and a number of extracellular enzymes and secondary metabolic pathway operons that distinguish these species. These differences include a region of more than 80 kilobases (kb) that comprises a cluster of polyketide synthase genes and a second operon of 38 kb encoding plipastatin synthase enzymes that are absent in the Bacillus licheniformis ATCC 14580 genome.

Public databases now contain a multitude of complete bacterial genomes, including several genomes from different strains of the same species. Recent analyses have shown, using pairwise whole genome alignments, that different strains of the same species may differ substantially in gene content. For example, genome comparisons of Escherichia coli strains CFT073, EDL933 and MG1655 revealed that only 39.2% of their combined set of proteins (gene products) are common to all three strains highlighting the astonishing diversity among strains of the same species (Welch et al., 2002, Proc. Nat. Acad. Sci. USA 99: 17020-17024; Perna et al., 2001, Nature 409: 529-533; Hayashi et al., 2001, DNA Res. 8: 11-22; Blattner et al., 1997, Science 277: 1453-1474). Furthermore, the genome sequence of E. coli strain CFT073 revealed 1,623 strain-specific genes (21.2%). From comparisons of this type, it is clearly seen that bacterial genomes are segmented into a common conserved backbone and strain-specific sequences. Typically the genome of a given strain within a species shows a mosaic structure in terms of the distribution of conserved “backbone” genes conserved among all strains and non-conserved genes that may have been acquired by horizontal transfer (Welch et al., 2002, Proc. Nat. Acad. Sci. USA 99: 17020-17024; Brzuszkiewicz et al., 2006, Proc. Nat. Acad. Sci. USA 103: 12879-12884).

Therefore, it would be advantageous to the art to have available the complete primary structure of the chromosomal DNA molecule of the Bacillus licheniformis strain SJ1904, an industrial strain that differs from the type strain ATCC 14580. With the complete chromosome data in hand, it should be possible to do comparative genomics and proteomics studies that can lead to improved industrial strains as well as to a better understanding of genome evolution among closely-related bacilli in the subtilis-licheniformis group.

### SUMMARY OF THE INVENTION

The present invention relates to isolated features (polynucleotides) of Bacillus licheniformis SJ1904 encoding biologically active substances selected from the group consisting of SEQ ID NOs: 1, 2, 3, 4, 5, 6 and subsequences thereof encoding fragments having biological activity.

The present invention also relates to isolated features (polynucleotides) encoding biologically active substances, selected from the group consisting of:

(a) a polynucleotide comprising a nucleotide sequence having preferably at least 60% identity, more preferably at least 65% identity, more preferably at least 70% identity, more preferably at least 75% identity, more preferably at least 80% identity, more preferably at least 85% identity, more preferably at least 90% identity, even more preferably at least 90% identity, most preferably at least 95% identity, and even most preferably at least 97% identity with a polynucleotide selected from the group consisting of SEQ ID NOs: 1, 2, 3, 4, 5, 6 and subsequences thereof encoding fragments having biological activity;

(b) a polynucleotide comprising a nucleotide sequence that hybridizes under preferably at least medium stringency conditions, more preferably at least medium-high stringency conditions, or most preferably at least high stringency conditions with a polynucleotide selected from the group consisting of SEQ ID NOs: 1, 2, 3, 4, 5, 6 and full-length complementary strands thereof;

(c) a polynucleotide encoding a biologically active substance comprising an amino acid sequence having preferably at least 60% identity, more preferably at least 65% identity, more preferably at least 70% identity, more preferably at least 75% identity, more preferably at least 80% identity, more preferably at least 85% identity, even more preferably at least 90% identity, most preferably at least 95% identity, and even most preferably at least 97% identity with an amino acid sequence selected from the group consisting of SEQ ID NOs: 7, 8, 9, 10, 11, 12 and fragments thereof retaining biological activity; and

(d) a polynucleotide encoding an artificial variant comprising a substitution, deletion, and/or insertion of one or more (several) amino acids of an amino acid sequence selected from the group consisting of SEQ ID NOs: 7, 8, 9, 10, 11, 12 and fragments thereof retaining biological activity.

The present invention also relates to nucleic acid constructs, vectors, and host cells comprising the isolated polynucleotides.

The present invention also relates to isolated biologically active substances comprising an amino acid sequence selected from the group consisting of SEQ ID NOs: 7, 8, 9, 10, 11, 12 and fragments thereof retaining biological activity.

The present invention also relates to isolated biologically active substances, selected from the group consisting of:

(a) a biologically active substance comprising an amino acid sequence having preferably at least 60% identity, more preferably at least 65% identity, more preferably at least 70% identity, more preferably at least 75% identity, more preferably at least 80% identity, more preferably at least 85% identity, even more preferably at least 90% identity, most preferably at least 95% identity, and even most preferably at least 97% identity with an amino acid sequence selected from the group consisting of SEQ ID NOs: 7, 8, 9, 10, 11, 12 and fragments thereof retaining biological activity;

(b) a biologically active substance encoded by a polynucleotide comprising a nucleotide sequence having preferably at least 60% identity, more preferably at least 65% identity, more preferably at least 70% identity, more preferably at least 75% identity, more preferably at least 80% identity, more preferably at least 85% identity, even more preferably at least 90% identity, most preferably at least 95% identity, and even most preferably at least 97% identity with a polynucleotide selected from the group consisting of SEQ ID NOs: 1, 2, 3, 4, 5, 6 and subsequences thereof encoding fragments having biological activity;

(c) a biologically active substance encoded by a polynucleotide comprising a nucleotide sequence that hybridizes under preferably at least medium stringency conditions, more preferably at least medium-high stringency conditions, or most preferably at least high stringency conditions with a polynucleotide selected from the group consisting of SEQ ID NOs: 1, 2, 3, 4, 5, 6 and full-length complementary strands thereof; and

(d) an artificial variant comprising a substitution, deletion, and/or insertion of one or more (several) amino acids of an amino acid sequence selected from the group consisting of SEQ ID NOs: 7, 8, 9, 10, 11, 12 and fragments thereof retaining biological activity.

The present invention also relates to methods for producing such substances having biological activity comprising (a) cultivating a recombinant host cell comprising a nucleic acid construct comprising a polynucleotide encoding the biologically active substance under conditions suitable for production of the biologically active substance; and (b) recovering the biologically active substance.

The present invention also relates to methods for monitoring differential expression of a plurality of genes in a first bacterial cell relative to expression of the same genes in one or more (several) second bacterial cells, comprising:

(a) adding a mixture of detection reporter-labeled nucleic acids isolated from the bacterial cells to a substrate containing an array of Bacillus licheniformis polynucleotides selected from the group consisting of SEQ ID NOs: 1, 2, 3, 4, 5, 6, full-length complementary strands of SEQ ID NOs: 1, 2, 3, 4, 5, 6, and fragments of SEQ ID NOs: 1, 2, 3, 4, 5, 6, under conditions where the detection reporter-labeled nucleic acids hybridize to complementary sequences of the Bacillus licheniformis polynucleotides on the array, wherein the nucleic acids from the first bacterial cell and the one or more (several) second bacterial cells are labeled with a first detection reporter and one or more (several) different second detection reporters, respectively; and

(b) examining the array under conditions wherein the relative expression of the genes in the bacterial cells is determined by the observed detection signal of each spot on the array in which (i) the Bacillus licheniformis polynucleotides on the array that hybridize to the nucleic acids obtained from either the first or the one or more (several) second bacterial cells produce a distinct first detection signal or one or more (several) second detection signals, respectively, and (ii) the Bacillus licheniformis polynucleotides on the array that hybridize to the nucleic acids obtained from both the first and one or more (several) second bacterial produce a distinct combined detection signal.

The present invention also relates to methods for isolating a polynucleotide encoding an enzyme, comprising:

(a) adding a mixture of labeled first nucleic acid probes, isolated from a microbial strain cultured on medium without an inducing substrate, and labeled second nucleic acid probes, isolated from the microbial strain cultured on medium with the inducing substrate, to an array of Bacillus licheniformis polynucleotides selected from the group consisting of the polynucleotides of SEQ ID NOs: 1, 2, 3, 4, 5, 6, full-length complementary strands of SEQ ID NOs: 1, 2, 3, 4, 5, 6, and fragments of SEQ ID NOs: 1, 2, 3, 4, 5, 6, under conditions where the labeled nucleic acid probes hybridize to complementary sequences of the Bacillus licheniformis polynucleotides on the array, wherein the first nucleic acid probes are labeled with a first reporter and the second nucleic acid probes are labeled with a second reporter;

(b) examining the array under conditions wherein the relative expression of the genes of the microbial strain is determined by the observed hybridization reporter signal of each spot on the array in which (i) the Bacillus licheniformis polynucleotides on the array that hybridize to the first nucleic acid probes produce a distinct first hybridization reporter signal or the second nucleic acid probes produce a distinct second hybridization reporter signal, and (ii) the Bacillus licheniformis polynucleotides on the array that hybridize to both the first and second nucleic acid probes produce a distinct combined hybridization reporter signal; and

(c) isolating a polynucleotide from the microbial strain that encodes an enzyme that degrades or converts the substrate.

The present invention also relates to genes or polynucleotides isolated by such methods and nucleic acid constructs, vectors, and host cells containing the isolated genes or polynucleotides.

### Definitions

Biologically active substance: The term “substance having biological activity” or “biologically active substance” is defined herein as any substance having biological activity encoded by a single gene or polynucleotide. Such substances include, but are not limited to, polypeptides (e.g., enzymes) and RNA (e.g., mRNA, tRNA, rRNA, and ncRNA). For purposes of the present invention, biological activity is determined according to procedures known in the art such as those described by Carpenter and Sabatini, 2004, Nature 5: 11-22; Sordie et al., 2003, Proceedings of the National Academy of Sciences USA 100: 11964-11969; Braun and LaBaer, 2003, TRENDS in Biotechnology 21: 383-388; and Kaberdin and McDowall, 2003, Genome Research 13: 1961-1965.

In a preferred aspect, the biologically active substance is a polypeptide. The polypeptide may be any polypeptide having a biological activity of interest. The term “polypeptide” is not meant herein to refer to a specific length of the encoded product and, therefore, encompasses peptides, oligopeptides, and proteins.

In a more preferred aspect, the polypeptide is an antibody, antigen, antimicrobial peptide, enzyme, growth factor, hormone, immunodilator, neurotransmitter, receptor, reporter protein, structural protein, transcription factor, and transporter.

In an even more preferred aspect, the polypeptide is an oxidoreductase, transferase, hydrolase, lyase, isomerase, or ligase.

In a most preferred aspect, the polypeptide is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, haloperoxidase, invertase, laccase, lipase, mannosidase, mutanase, oxidase, pectinolytic enzyme, peptidoglutaminase, peroxidase, phospholipase, phytase, polyphenoloxidase, proteolytic enzyme, ribonuclease, transglutaminase, urokinase, or xylanase

In another more preferred aspect, the polypeptide is an albumin, collagen, tropoelastin, elastin, or gelatin.

Isolated biologically active substance: The term “isolated biologically active substance” is defined herein as a substance that is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE, HPLC, capillary electrophoresis, or any other method used in the art.

Substantially pure biologically active substance or pure biologically active substance: The term “substantially pure biologically active substance” is defined herein as a biologically active substance preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other material with which it is natively associated. It is, therefore, preferred that the substantially pure biologically active substance is at least 92% pure, preferably at least 94% pure, more preferably at least 95% pure, more preferably at least 96% pure, more preferably at least 96% pure, more preferably at least 97% pure, even more preferably at least 98% pure, most preferably at least 99%, and even most preferably at least 99.5% pure by weight of the total material present in the preparation. The term “pure biologically active substance” is defined as a biologically active substance preparation that contains no other material with which it is natively associated. The biologically active substances of the present invention are preferably in a substantially pure form. In particular, it is preferred that the biologically active substances are in “essentially pure form”, i.e., that the biologically active substance preparation is essentially free of other material with which it is natively or recombinantly associated. This can be accomplished, for example, by preparing the biologically active substance by means of well-known recombinant methods or by classical purification methods.

Identity: The relatedness between two amino acid sequences or between two nucleotide sequences is described by the parameter “identity”.

For purposes of the present invention, the degree of sequence identity between two amino acid sequences is determined by the Smith-Waterman Protein method for the GENEMATCHER2™ as implemented by Paracel Inc. (Pasadena, Calif.), or the BLASTP method as described by Altschul et al., 1990, Journal of Molecular Biology 215: 403-410.

For purposes of the present invention, the degree of sequence identity between two nucleotide sequences is determined by the Smith Waterman nucleotide method for the GENEMATCHER2™ or BLASTN for the BlastMachine as implemented by Paracel Inc.

Polypeptide Fragment: The term “polypeptide fragment” is defined herein as a polypeptide, which retains biological activity, having one or more (several) amino acids deleted from the amino and/or carboxyl terminus of a polypeptide encoded by any of the polynucleotides of the present invention, i.e., polypeptides of SEQ ID NOs: 7, 8, 9, 10, 11, 12. Preferably, a fragment contains at least 80%, preferably at least 85%, more preferably at least 90%, even more preferably at least 95%, and most preferably at least 97% of the amino acid residues of the mature polypeptide product.

Subsequence: The term “subsequence” is defined herein as a polynucleotide comprising a nucleotide sequence of any of SEQ ID NOs: 1, 2, 3, 4, 5, 6 except that one or more (several) nucleotides have been deleted from the 5′ and/or 3′ end. Preferably, a subsequence contains at least 80%, preferably at least 85%, more preferably at least 90%, even more preferably at least 95%, and most preferably at least 97% of the nucleotides of any of the isolated polynucleotides of the present invention.

Substantially pure polynucleotide or pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof. The term “pure polynucleotide” is defined as a polynucleotide preparation that contains no other material with which it is natively associated.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a biologically active substance of the present invention. Each control sequence may be native or foreign to the polynucleotide encoding the substance. Such control sequences include, but are not limited to, a leader, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a biologically active substance.

Operably linked: The term “operably linked” as used herein refers to a configuration in which a control sequence is placed at an appropriate position relative to the coding sequence of the DNA sequence, such that the control sequence directs the expression of a biologically active substance.

Coding sequence: When used herein the term “coding sequence” is intended to cover a nucleotide sequence, which directly specifies the amino acid sequence of its protein product. The boundaries of the coding sequence are generally determined by an open reading frame, which usually begins with the ATG start codon or alternative start codons such as GTG and TTG.

Expression: The term “expression” includes any step involved in the production of a biologically active substance including, but not limited to, transcription, post-transcriptional modification, translation, post-translational modification, and secretion.

Expression vector: The term “expression vector” herein covers a DNA molecule, linear or circular, that comprises a segment encoding a biologically active substance of the invention, and is operably linked to additional segments that provide for its transcription.

Host cell: The term “host cell”, as used herein, includes any cell type that is susceptible to transformation, transfection, conjugation, electroporation, etc. with a nucleic acid construct, plasmid, or vector.

Modification: The term “modification” means herein any chemical modification of a biological substance, e.g., polypeptide, as well as genetic manipulation of the DNA encoding that biological substance. The modification can be a substitution, a deletion and/or an insertion of one or more (several) amino acids as well as replacements of one or more (several) amino acid side chains.

Artificial variant: When used herein, the term “artificial variant” means a polypeptide having biological activity produced by an organism expressing a modified nucleotide sequence, or the mature polypeptide coding region thereof. The modified nucleotide sequence is obtained through human intervention by modification of the nucleotide sequence disclosed or a homologous sequence thereof, or the mature polypeptide coding region thereof.

### DETAILED DESCRIPTION OF THE INVENTION

Bacillus licheniformis SJ1904 and Features (Polynucleotides) Thereof

Bacillus licheniformis SJ1904 consists of a circular molecule of 4,345,159 base pairs with a mean % G+C content of 46.7%. The chromosome contains predicted protein-coding genes (SEQ ID NOs: 1, 2, 3, 4, 5, 6). The deduced amino acid sequences of the predicted protein-coding genes are shown in SEQ ID NOs: 7, 8, 9, 10, 11, 12. SEQ ID NO: 7 corresponds to SEQ ID NO: 1, SEQ ID NO: 8 corresponds to SEQ ID NO: 2, SEQ ID NO: 9 corresponds to SEQ ID NO: 3, etc. The predicted functions of the gene products are shown in Table 1.

The present invention also relates to isolated features (polynucleotides) of the complete chromosomal DNA molecule of Bacillus licheniformis SJ1904 encoding biologically active substances, selected from the group consisting of SEQ ID NOs: 1, 2, 3, 4, 5, 6 and subsequences thereof encoding fragments having biological activity.

The present invention also relates to isolated features (polynucleotides) encoding biologically active substances, selected from the group consisting of:

(a) a polynucleotide comprising a nucleotide sequence having preferably at least 60% identity, more preferably at least 65% identity, more preferably at least 70% identity, more preferably at least 75% identity, more preferably at least 80% identity, more preferably at least 85% identity, even more preferably at least 90% identity, most preferably at least 95% identity, and even most preferably at least 97% identity with a polynucleotide selected from the group consisting of SEQ ID NOs: 1, 2, 3, 4, 5, 6 and subsequences thereof encoding fragments having biological activity;

(b) a polynucleotide comprising a nucleotide sequence that hybridizes under preferably at least medium stringency conditions, more preferably at least medium-high stringency conditions, or most preferably at least high stringency conditions with a polynucleotide selected from the group consisting of SEQ ID NOs: 1, 2, 3, 4, 5, 6 and full-length complementary strands thereof;

(c) a polynucleotide encoding a biologically active substance comprising an amino acid sequence having preferably at least 60% identity, more preferably at least 65% identity, more preferably at least 70% identity, more preferably at least 75% identity, more preferably at least 80% identity, more preferably at least 85% identity, even more preferably at least 90% identity, most preferably at least 95% identity, and even most preferably at least 97% identity with an amino acid sequence selected from the group consisting of SEQ ID NOs: 7, 8, 9, 10, 11, 12 and fragments thereof retaining biological activity; and

(d) a polynucleotide encoding an artificial variant comprising a substitution, deletion, and/or insertion of one or more (several) amino acids of an amino acid sequence selected from the group consisting of SEQ ID NOs: 7, 8, 9, 10, 11, 12 and fragments thereof retaining biological activity.

In a first aspect, the present invention relates to an isolated polynucleotide having a degree of sequence identity to a polynucleotide selected from the group consisting of SEQ ID NOs: 1, 2, 3, 4, 5, 6 of at least 60%, preferably at least 65%, more preferably at least 70%, more preferably at least 75%, more preferably at least 80%, more preferably at least 85%, even more preferably at least 90%, most preferably at least 95%, and even most preferably at least 96%, 97%, 98%, or 99%, which encode biologically active substances having a particular biological activity (hereinafter “homologous biologically active substances”).

In a second aspect, the present invention relates to an isolated polynucleotide comprising a nucleotide sequence that hybridizes under very low stringency conditions, preferably low stringency conditions, more preferably medium stringency conditions, more preferably medium-high stringency conditions, even more preferably high stringency conditions, and most preferably very high stringency conditions with any of (i) the polynucleotides of SEQ ID NOs: 1, 2, 3, 4, 5, 6, or subsequences thereof, or (ii) full-length complementary strands thereof (J. Sambrook, E. F. Fritsch, and T. Maniatus, 1989, Molecular Cloning, A Laboratory Manual, 2d edition, Cold Spring Harbor, N.Y.). Subsequences of SEQ ID NOs: 1, 2, 3, 4, 5, 6 may be preferably at least 90 nucleotides, more preferably at least 150 nucleotide, and most preferably at least 200 nucleotides. Moreover, the subsequences may encode fragments of a gene product that have biological activity.

In a preferred aspect, the present invention relates to an isolated polynucleotide comprising a nucleotide sequence that hybridizes under preferably at least medium stringency conditions, more preferably at least medium-high stringency conditions, or most preferably at least high stringency conditions with a polynucleotide selected from the group consisting of SEQ ID NOs: 1, 2, 3, 4, 5, 6 and full-length complementary strands thereof.

The nucleotide sequences of SEQ ID NOs: 1, 2, 3, 4, 5, 6 or subsequences thereof, as well as the amino acid sequences of SEQ ID NOs: 7, 8, 9, 10, 11, 12 or fragments thereof, may be used to design nucleic acid probes to identify and clone DNA encoding biologically active substances from strains of different genera or species according to methods well known in the art. In particular, such probes can be used for hybridization with the genomic DNA of the genus or species of interest, following standard Southern blotting procedures, in order to identify and isolate the corresponding gene therein. Such probes can be considerably shorter than the entire sequence, but should be at least 14, preferably at least 25, more preferably at least 35 nucleotides in length, such as at least 70 nucleotides in length. It is preferred, however, that the nucleic acid probes are at least 100 nucleotides in length. For example, the nucleic acid probes may be at least 200 nucleotides, at least 300 nucleotides, at least 400 nucleotides, or at least 500 nucleotides in length. Even longer probes may be used, e.g., nucleic acid probes that are at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA library prepared from such other organisms may, therefore, be screened for DNA that hybridizes with the probes described above and encodes a biologically active substance. Genomic DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with any of SEQ ID NOs: 1, 2, 3, 4, 5, 6 or subsequences thereof, the carrier material is used in a Southern blot.

For purposes of the present invention, hybridization indicates that a polynucleotide hybridizes to a labeled gene having the nucleotide sequence shown in any of SEQ ID NOs: 1, 2, 3, 4, 5, 6, full-length complementary strands thereof, or subsequences thereof, under very low to very high stringency conditions. Molecules to which the nucleic acid probe hybridizes under these conditions can be detected using X-ray film.

In a preferred aspect, the nucleic acid probe is any of the polynucleotides of SEQ ID NOs: 1, 2, 3, 4, 5, 6, or subsequences thereof. In another preferred aspect, the nucleic acid probe is the mature polypeptide coding region of any of the polynucleotides of SEQ ID NOs: 1, 2, 3, 4, 5, 6. In another preferred aspect, the nucleic acid probe is the polynucleotide of any of SEQ ID NOs: 1, 2, 3, 4, 5, 6 contained in chromosome of Bacillus licheniformis SJ1904. In another preferred aspect, the nucleic acid probe is the mature polypeptide coding region of any of the polynucleotides of SEQ ID NOs: 1, 2, 3, 4, 5, 6 contained in Bacillus licheniformis SJ1904.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes of about 14 nucleotides to about 70 nucleotides in length, stringency conditions are defined as prehybridization, hybridization, and washing post-hybridization at about 5° C. to about 10° C. below the calculated Tₘ, using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

For short probes of about 14 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Under salt-containing hybridization conditions, the effective Tₘ is what controls the degree of sequence identity required between the probe and the filter bound DNA for successful hybridization. The effective Tₘ may be determined using the formula below to determine the degree of sequence identity required for two DNAs to hybridize under various stringency conditions.

Effective Tₘ=81.5+16.6(log M[Na⁺])+0.41(% G+C)−0.72(% formamide)

The % G+C content of any of the polynucleotides of SEQ ID NOs: 1, 2, 3, 4, 5, 6 can easily be determined. For medium stringency, for example, the concentration of formamide is 35% and the Na⁺ concentration for 5×SSPE is 0.75 M. Applying this formula to these values, the Effective Tₘ in ° C. can be calculated. Another relevant relationship is that a 1% mismatch of two DNAs lowers the Tₘ 1.4° C. To determine the degree of sequence identity required for two DNAs to hybridize under medium stringency conditions at 42° C., the following formula is used:

% Homology=100−[(Effective Tₘ−Hybridization Temperature)/1.4]

Applying this formula, the degree of sequence identity required for two DNAs to hybridize under medium stringency conditions at 42° C. can be calculated.

Similar calculations can be made under other stringency conditions, as defined herein.

The present invention also relates to isolated polynucleotides obtained by (a) hybridizing a population of DNA under very low, low, medium, medium-high, high, or very high stringency conditions with any of (i) the polynucleotides of SEQ ID NOs: 1, 2, 3, 4, 5, 6, or subsequences thereof, or (ii) full-length complementary strands thereof; and (b) isolating the hybridizing polynucleotide from the population of DNA. In a preferred aspect, the hybridizing polynucleotide encodes a polypeptide of any of SEQ ID NOs: 1, 2, 3, 4, 5, 6, or homologous polypeptides thereof.

In a preferred aspect, the present invention relates to isolated polynucleotides obtained by (a) hybridizing a population of DNA under preferably at least medium stringency conditions, more preferably at least medium-high stringency conditions, or most preferably at least high stringency conditions with a polynucleotide selected from the group consisting of SEQ ID NOs: 1, 2, 3, 4, 5, 6 and full-length complementary strands thereof; and (b) isolating the hybridizing polynucleotide from the population of DNA.

In a third aspect, the present invention relates to an isolated polynucleotide encoding a biologically active substance comprising an amino acid sequence having preferably at least 60% identity, more preferably at least 65% identity, more preferably at least 70% identity, more preferably at least 75% identity, more preferably at least 80% identity, more preferably at least 85% identity, more preferably at least 90% identity, even more preferably at least 90% identity, most preferably at least 95% identity, and even most preferably at least 97% identity with an amino acid sequence selected from the group consisting of SEQ ID NOs: 7, 8, 9, 10, 11, 12 and fragments thereof retaining biological activity.

In a fourth aspect, the present invention relates to a polynucleotide encoding an artificial variant comprising a substitution, deletion, and/or insertion of one or more (several) amino acids of any of SEQ ID NOs: 7, 8, 9, 10, 11, 12 or a homologous sequence thereof; or the mature polypeptide thereof. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to a polypeptide according to the invention.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of any of SEQ ID NOs: 7, 8, 9, 10, 11, 12 is 10, preferably 9, more preferably 8, more preferably 7, more preferably at most 6, more preferably 5, more preferably 4, even more preferably 3, most preferably 2, and even most preferably 1.

### Biologically Active Substances

The present invention also relates to isolated biologically active substances, selected from the group consisting of:

(a) a biologically active substance comprising an amino acid sequence having preferably at least 60% identity, more preferably at least 65% identity, more preferably at least 70% identity, more preferably at least 75% identity, more preferably at least 80% identity, more preferably at least 85% identity, more preferably at least 90% identity, even more preferably at least 90% identity, most preferably at least 95% identity, and even most preferably at least 97% identity with an amino acid sequence selected from the group consisting of SEQ ID NOs: 7, 8, 9, 10, 11, 12 and fragments thereof retaining biological activity;

(b) a biologically active substance encoded by a polynucleotide comprising a nucleotide sequence having preferably at least 60% identity, more preferably at least 65% identity, more preferably at least 70% identity, more preferably at least 75% identity, more preferably at least 80% identity, more preferably at least 85% identity, more preferably at least 90% identity, even more preferably at least 90% identity, most preferably at least 95% identity, and even most preferably at least 97% identity with a polynucleotide selected from the group consisting of SEQ ID NOs: 1, 2, 3, 4, 5, 6 and subsequences thereof encoding fragments having biological activity;

(c) a biologically active substance encoded by a polynucleotide comprising a nucleotide sequence that hybridizes under preferably at least medium stringency conditions, more preferably at least medium-high stringency conditions, or most preferably at least high stringency conditions with a polynucleotide selected from the group consisting of SEQ ID NOs: 1, 2, 3, 4, 5, 6 and full-length complementary strands thereof; and

(d) an artificial variant comprising a substitution, deletion, and/or insertion of one or more (several) amino acids of an amino acid sequence selected from the group consisting of SEQ ID NOs: 7, 8, 9, 10, 11, 12 and fragments thereof retaining biological activity.

In a first aspect, the present invention also relates to an isolated biologically active substance comprising an amino acid sequence having a degree of sequence identity to any of SEQ ID NOs: 7, 8, 9, 10, 11, 12 of at least 60%, preferably at least 65%, more preferably at least 70%, more preferably at least 75%, more preferably at least 80%, more preferably at least 85%, even more preferably at least 90%, most preferably at least 95%, and even most preferably at least 97%, which have biological activity (hereinafter “homologous polypeptides”). In a preferred aspect, the homologous polypeptides have an amino acid sequence that differs by ten amino acids, preferably by five amino acids, more preferably by four amino acids, even more preferably by three amino acids, most preferably by two amino acids, and even most preferably by one amino acid from the amino acid sequences of SEQ ID NOs: 7, 8, 9, 10, 11, 12.

A biologically active substance preferably comprises the amino acid sequence of any of SEQ ID NOs: 7, 8, 9, 10, 11, 12; or fragments thereof that have biological activity. In a more preferred aspect, a biologically active substance comprises the amino acid sequence of any of SEQ ID NOs: 7, 8, 9, 10, 11, 12. In another preferred aspect, a biologically active substance comprises the mature polypeptide region of any of SEQ ID NOs: 7, 8, 9, 10, 11, 12, or fragments thereof that have biological activity. In another preferred aspect, a biologically active substance comprises the mature polypeptide region of any of SEQ ID NOs: 7, 8, 9, 10, 11, 12. In another preferred aspect, a biologically active substance consists of the amino acid sequence of any of SEQ ID NOs: 7, 8, 9, 10, 11, 12; or fragments thereof that have biological activity. In another preferred aspect, a biologically active substance consists of the amino acid sequence of any of SEQ ID NOs: 7, 8, 9, 10, 11, 12. In another preferred aspect, a biologically active substance consists of the mature polypeptide region of any of SEQ ID NOs: 7, 8, 9, 10, 11, 12; or fragments thereof that have biological activity. In another preferred aspect, a biologically active substance consists of the mature polypeptide region of any of SEQ ID NOs: 7, 8, 9, 10, 11, 12.

In a second aspect, the present invention relates to a biologically active substance encoded by a polynucleotide comprising a nucleotide sequence having preferably at least 60% identity, more preferably at least 65% identity, more preferably at least 70% identity, more preferably at least 75% identity, more preferably at least 80% identity, more preferably at least 85% identity, more preferably at least 90% identity, even more preferably at least 90% identity, most preferably at least 95% identity, and even most preferably at least 97% identity with a polynucleotide selected from the group consisting of SEQ ID NOs: 1, 2, 3, 4, 5, 6 and subsequences thereof encoding fragments having biological activity.

In a third aspect, the present invention relates to an isolated biologically active substance encoded by a polynucleotide that hybridizes, as described above, under very low stringency conditions, preferably low stringency conditions, more preferably medium stringency conditions, more preferably medium-high stringency conditions, even more preferably high stringency conditions, and most preferably very high stringency conditions with a polynucleotide selected from the group consisting of (i) the polynucleotides of SEQ ID NOs: 1, 2, 3, 4, 5, 6, and subsequences thereof, and (ii) full-length complementary strands thereof. A subsequence of any of SEQ ID NOs: 1, 2, 3, 4, 5, 6 may be at least 100 nucleotides or preferably at least 200 nucleotides. Moreover, the subsequence may encode a fragment, e.g., a fragment that has biological activity.

In a fourth aspect, the present invention relates to an artificial variant comprising a substitution, deletion, and/or insertion of one or more (several) amino acids of an amino acid sequence selected from the group consisting of SEQ ID NOs: 7, 8, 9, 10, 11, 12 and fragments thereof retaining biological activity, as described above.

### Nucleic Acid Constructs

The present invention also relates to nucleic acid constructs comprising an isolated polynucleotide or isolated polynucleotides (e.g., operon) of the present invention operably linked to one or more (several) control sequences that direct the expression of the coding sequence in a suitable host cell under conditions compatible with the control sequences.

An isolated polynucleotide(s) of the present invention may be manipulated in a variety of ways to provide for production of a biologically active substance encoded directly or indirectly by the polynucleotide(s). Manipulation of the nucleotide sequence prior to its insertion into a vector may be desirable or necessary depending on the expression vector. The techniques for modifying nucleotide sequences utilizing recombinant DNA methods are well known in the art.

The control sequence may be an appropriate promoter sequence, a nucleotide sequence that is recognized by a host cell for expression of the polynucleotide(s) encoding the biologically active substance. The promoter sequence contains transcriptional control sequences that mediate the expression of the biologically active substance. The promoter may be any nucleotide sequence that shows transcriptional activity in the host cell of choice including mutant, truncated, and hybrid promoters, and may be obtained from genes encoding extracellular or intracellular polypeptides or biologically active substances either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

The control sequence may also be a suitable transcription terminator sequence, a sequence recognized by a host cell to terminate transcription. The terminator sequence is operably linked to the 3′ terminus of the gene encoding the biologically active substance. Any terminator that is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region that directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE) and Bacillus subtilis neutral protease (nprT).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences that allow the regulation of the expression of a biologically active substance relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. Other examples of regulatory sequences are those that allow for gene amplification. In eukaryotic systems, these include the dihydrofolate reductase gene that is amplified in the presence of methotrexate, and the metallothionein genes that are amplified with heavy metals. In these cases, the nucleotide sequence encoding the biologically active substance would be operably linked with the regulatory sequence.

### Expression Vectors

The present invention also relates to recombinant expression vectors comprising an isolated polynucleotide of the present invention, a promoter, and transcriptional and translational stop signals. The various nucleic acid and control sequences described above may be joined together to produce a recombinant expression vector that may include one or more (several) convenient restriction sites to allow for insertion or substitution of the nucleotide sequence encoding the polypeptide at such sites. Alternatively, a polynucleotide of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about the expression of a polynucleotide of the present invention. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon may be used.

The vectors of the present invention preferably contain one or more (several) selectable markers that permit easy selection of transformed cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers that confer antibiotic resistance such as ampicillin, kanamycin, chloramphenicol or tetracycline resistance.

The vectors of the present invention preferably contain an element(s) that permits integration of the vector into the host cell's genome or autonomous replication of the vector in the cell independent of the genome.

For integration into the host cell genome, the vector may rely on portions of the sequence of the gene or any other element of the vector for integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a sequence that enables a plasmid or vector to replicate in vivo. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide of the present invention may be inserted into the host cell to increase production of the polynucleotide product. An increase in the copy number of the polynucleotide can be obtained by integrating at least one additional copy of the sequence into the host cell genome or by including an amplifiable selectable marker gene with a polynucleotide of the present invention where cells containing amplified copies of the selectable marker gene, and thereby additional copies of the polynucleotide of the present invention, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising an isolated polynucleotide of the present invention, where the host cells are advantageously used in the recombinant production of a biologically active substance encoded by the polynucleotide. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the polynucleotide encoding the biologically active substance and its source.

The host cell may be any unicellular microorganism, e.g., a prokaryote, or a non-unicellular microorganism, e.g., a eukaryote.

The bacterial host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In a preferred aspect, the bacterial host cell is a Bacillus amyloliquefaciens, Bacillus lentus, Bacillus licheniformis, Bacillus stearothermophilus or Bacillus subtilis cell. In a more preferred aspect, the bacterial host cell is a Bacillus amyloliquefaciens cell. In another more preferred aspect, the bacterial host cell is a Bacillus clausii cell. In another more preferred aspect, the bacterial host cell is a Bacillus licheniformis cell. In another more preferred aspect, the bacterial host cell is a Bacillus subtilis cell.

The bacterial host cell may also be any Streptococcus cell. Streptococcus cells useful in the practice of the present invention include, but are not limited to, Streptococcus equisimilis, Streptococcus pyogenes, Streptococcus uberis, and Streptococcus equi subsp. Zooepidemicus.

In a preferred aspect, the bacterial host cell is a Streptococcus equisimilis cell. In another preferred aspect, the bacterial host cell is a Streptococcus pyogenes cell. In another preferred aspect, the bacterial host cell is a Streptococcus uberis cell. In another preferred aspect, the bacterial host cell is a Streptococcus equi subsp. Zooepidemicus cell.

The bacterial host cell may also be any Streptomyces cell. Streptomyces cells useful in the practice of the present invention include, but are not limited to, Streptomyces achromogenes, Streptomyces avermitilis, Streptomyces coelicolor, Streptomyces griseus, and Streptomyces lividans.

In a preferred aspect, the bacterial host cell is a Streptomyces achromogenes cell. In another preferred aspect, the bacterial host cell is a Streptomyces avermitilis cell. In another preferred aspect, the bacterial host cell is a Streptomyces coelicolor cell. In another preferred aspect, the bacterial host cell is a Streptomyces griseus cell. In another preferred aspect, the bacterial host cell is a Streptomyces lividans cell.

The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). The introduction of DNA into an E coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98:6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios. 68: 189-2070, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the for introducing DNA into a host cell can be used.

### Methods of Production

The present invention also relates to methods for producing a biologically active substance of the present invention comprising (a) cultivating a strain, which in its wild-type form is capable of producing the biologically active substance, under conditions conducive for production of the biologically active substance; and (b) recovering the biologically active substance. Preferably, the strain is of the genus Bacillus, and more preferably Bacillus licheniformis.

The present invention also relates to methods for producing a biologically active substance of the present invention comprising (a) cultivating a host cell under conditions conducive for production of the biologically active substance; and (b) recovering the biologically active substance.

The present invention also relates to methods for producing a biologically active substance of the present invention comprising (a) cultivating a host cell under conditions conducive for production of the biologically active substance, wherein the host cell comprises a mutant polynucleotide comprising at least one mutation in the coding region of any of SEQ ID NOs: 1, 2, 3, 4, 5, 6, wherein the mutant polynucleotide encodes a biologically active substance that consists of SEQ ID NOs: 7, 8, 9, 10, 11, 12, respectively, and (b) recovering the biologically active substance.

In the production methods of the present invention, the cells are cultivated in a nutrient medium suitable for production of the biologically active substance using methods known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the biologically active substance to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the biologically active substance is secreted into the nutrient medium, the biologically active substance can be recovered directly from the medium. If the biologically active substance is not secreted, it can be recovered from cell lysates.

The biologically active substances may be detected using methods known in the art that are specific for the polypeptides. These detection methods may include use of specific antibodies, formation of an enzyme product, or disappearance of an enzyme substrate. For example, an enzyme assay may be used to determine the activity of an enzyme. See, for example, Enzyme Nomenclature, Academic Press, Inc., New York, 2007.

The resulting biologically active substances may be recovered by methods known in the art. For example, the biologically active substances may be recovered from the nutrient medium by conventional procedures including, but not limited to, centrifugation, filtration, extraction, spray-drying, evaporation, or precipitation.

The biologically active substances of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Removal or Reduction of Biologically Active Substance

The present invention also relates to methods for producing a mutant of a parent cell, which comprises disrupting or deleting all or a portion of a polynucleotide encoding a biologically active substance of the present invention, which results in the mutant cell producing less of the biologically active substance than the parent cell when cultivated under the same conditions.

The mutant cell may be constructed by reducing or eliminating expression of a gene encoding or regulatory synthesis of a biologically active substance of the present invention using methods well known in the art, for example, insertions, disruptions, replacements, or deletions. The gene to be modified or inactivated may be, for example, the coding region or a part thereof essential for activity, or a regulatory element of the gene required for the expression of the coding region. An example of such a regulatory or control sequence may be a promoter sequence or a functional part thereof, i.e., a part that is sufficient for affecting expression of the gene. Other control sequences for possible modification include, but are not limited to, a leader, propeptide sequence, signal peptide sequence, transcription terminator, and transcriptional activator.

Modification or inactivation of the gene may be performed by subjecting the parent cell to mutagenesis and selecting for mutant cells in which expression of the gene has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the nucleotide sequence may be accomplished by introduction, substitution, or removal of one or more (several) nucleotides in the gene or a regulatory element required for the transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the nucleotide sequence to be modified, it is preferred that the modification be performed in vitro as exemplified below.

An example of a convenient way to eliminate or reduce expression of a nucleotide sequence by a cell of choice is based on techniques of gene replacement, gene deletion, or gene disruption. For example, in the gene disruption method, a nucleic acid sequence corresponding to the endogenous nucleotide sequence is mutagenized in vitro to produce a defective nucleic acid sequence that is then transformed into the parent cell to produce a defective gene. By homologous recombination, the defective nucleic acid sequence replaces the endogenous nucleotide sequence. It may be desirable that the defective nucleotide sequence also encodes a marker that may be used for selection of transformants in which the nucleotide sequence has been modified or destroyed. In a particularly preferred aspect, the nucleotide sequence is disrupted with a selectable marker such as those described herein.

Alternatively, modification or inactivation of the nucleotide sequence may be performed by established anti-sense or RNA interference (RNAi) techniques using a sequence complementary to the nucleotide sequence. More specifically, expression of the nucleotide sequence by a cell may be reduced or eliminated by introducing a sequence complementary to the nucleic acid sequence of the gene that may be transcribed in the cell and is capable of hybridizing to the mRNA produced in the cell. Under conditions allowing the complementary anti-sense nucleotide sequence to hybridize to the mRNA, the amount of protein translated is thus reduced or eliminated.

The present invention further relates to a mutant cell of a parent cell that comprises a disruption or deletion of a nucleotide sequence encoding the biologically active substance or a control sequence thereof, which results in the mutant cell producing less of the biologically active substance than the parent cell.

The biologically active substance-deficient mutant cells so created are particularly useful as host cells for the expression of homologous and/or heterologous substances, such as polypeptides. Therefore, the present invention further relates to methods for producing a homologous or heterologous substance comprising (a) cultivating the mutant cell under conditions conducive for production of the substance; and (b) recovering the substance. The term “heterologous substances” is defined herein as substances that are not native to the host cell, a native substance in which modifications have been made to alter the native sequence, or a native substance whose expression is quantitatively altered as a result of a manipulation of the host cell by recombinant DNA techniques.

In a further aspect, the present invention relates to a method for producing a protein product essentially free of a biologically active substance by fermentation of a cell that produces both a biologically active substance of the present invention as well as the protein product of interest by adding an effective amount of an agent capable of inhibiting activity of the biologically active substance to the fermentation broth before, during, or after the fermentation has been completed, recovering the product of interest from the fermentation broth, and optionally subjecting the recovered product to further purification.

In accordance with this aspect of the invention, it is possible to remove at least 60%, preferably at least 75%, more preferably at least 85%, still more preferably at least 95%, and most preferably at least 99% of the biologically active substance. Complete removal of biologically active substance may be obtained by use of this method.

The methods used for cultivation and purification of the product of interest may be performed by methods known in the art.

The methods of the present invention for producing an essentially biologically active substance-free product is of particular interest in the production of prokaryotic polypeptides, in particular bacterial proteins such as enzymes. The enzyme may be selected from, e.g., an amylolytic enzyme, lipolytic enzyme, proteolytic enzyme, cellulytic enzyme, oxidoreductase, or plant cell-wall degrading enzyme. Examples of such enzymes include an aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinolytic enzyme, peroxidase, phytase, phenoloxidase, polyphenoloxidase, proteolytic enzyme, ribonuclease, transferase, transglutaminase, or xylanase. The biologically active substance-deficient cells may also be used to express heterologous proteins of pharmaceutical interest such as hormones, growth factors, receptors, polymers, e.g., hyaluronic acid and elastin, and the like.

It will be understood that the term “prokaryotic polypeptides” includes not only native polypeptides, but also those polypeptides, e.g., enzymes, which have been modified by amino acid substitutions, deletions or additions, or other such modifications to enhance activity, thermostability, pH tolerance and the like.

In a further aspect, the present invention relates to a product of a protein or substance essentially free of a biologically active substance of the invention, produced by a method of the present invention.

### Methods of Inhibiting Expression of a Polypeptide

The present invention also relates to methods of inhibiting the expression of a biological substance in a cell, comprising administering to the cell or expressing in the cell a double-stranded RNA (dsRNA) molecule, wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA (siRNAs) for inhibiting transcription. In another preferred aspect, the dsRNA is micro RNA (miRNAs) for inhibiting translation.

The present invention also relates to such double-stranded RNA (dsRNA) molecules, comprising a portion of the coding sequence of any of SEQ ID NOs: 1, 2, 3, 4, 5, 6 for inhibiting expression of a biological substance in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi).

The dsRNAs of the present invention can be used in gene-silencing therapeutics. In one aspect, the invention provides methods to selectively degrade RNA using the dsRNAis of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art, see, for example, U.S. Pat. No. 6,506,559; U.S. Pat. No. 6,511,824; U.S. Pat. No. 6,515,109; and U.S. Pat. No. 6,489,127.

### Compositions

The present invention also relates to compositions comprising a biologically active substance of the present invention. Preferably, the compositions are enriched in the biologically active substance. The term “enriched” indicates that the biologically active substance of the composition has been increased, e.g., with an enrichment factor of 1.1.

The composition may comprise a biologically active substance of the invention as the major component, e.g., a mono-component composition. Alternatively, the composition may comprise multiple biologically active substances, for example, multiple enzymes, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. For instance, the composition may be in the form of a granulate or a microgranulate. The biologically active substance to be included in the composition may be stabilized in accordance with methods known in the art.

Methods for Using the Bacillus licheniformis Sequences

The genes encoded in the chromosome may be used for monitoring global gene expression during the life cycle of the organism or during industrial fermentations (e.g., implemented on DNA microarrays). By monitoring global gene expression, for example, improved processes for industrial fermentation can be implemented with greater efficiency and economy.

### Methods for Isolating Genes

The present invention also relates to methods for isolating a polynucleotide encoding a biologically active substance from a microbial strain. The method comprises first the addition of a mixture of first labeled nucleic acid probes, isolated from a microbial strain cultured on medium without an inducing substrate, and a mixture of second labeled nucleic acid probes, isolated from the microbial strain cultured on medium with the inducing substrate, to an array of Bacillus licheniformis polynucleotides selected from the group consisting of the polynucleotides of SEQ ID NOs: 1, 2, 3, 4, 5, 6, full-length complementary strands of SEQ ID NOs: 1, 2, 3, 4, 5, 6, and fragments of SEQ ID NOs: 1, 2, 3, 4, 5, 6, under conditions where the labeled nucleic acid probes hybridize to complementary sequences of the Bacillus licheniformis polynucleotides on the array. The first nucleic acid probes are labeled with a first reporter and the second nucleic acid probes are labeled with a second reporter. The array is then examined under conditions wherein the relative expression of the genes of the microbial strain is determined by the observed hybridization reporter signal of each spot on the array in which (i) the Bacillus licheniformis polynucleotides on the array that hybridize to the first nucleic acid probes produce a distinct first hybridization reporter signal or to the second nucleic acid probes produce a distinct second hybridization reporter signal, and (ii) the Bacillus licheniformis polynucleotides on the array that hybridize to both the first and second nucleic acid probes produce a distinct combined hybridization reporter signal. The probe is then sequenced to isolate from the microbial strain the corresponding gene that encodes an enzyme that degrades or converts the substrate.

Enzymes. The gene of interest may encode any enzyme including an oxidoreductase, transferase, hydrolase, lyase, isomerase, or ligase. In a preferred aspect, the enzyme is an acylase, alpha-glucosidase, amidase, aminopeptidase, amylase, carbohydrase, carboxypeptidase, catalase, cellulase, chitinase, cutinase, cyclodextrin glycosyltransferase, deoxyribonuclease, dextrinase, endoglucanase, esterase, galactanase, alpha-galactosidase, beta-galactosidase, glucoamylase, glucanase, glucocerebrosidase, alpha-glucosidase, beta-glucosidase, hemicellulase, invertase, laccase, lignase, lipase, lysin, mannosidase, mutanase, oxidase, pectinolytic enzyme, peroxidase, phosphatase, phospholipase, phytase, polyphenoloxidase, proteolytic enzyme, pullulanase, ribonuclease, transglutaminase, urokinase, or xylanase.

Inducing Substrate. The inducing substrate may be any substrate that is subject to the action of an enzyme, i.e., that degrades or converts the substrate. In a preferred aspect, the inducing substrate is lignin or a lignin-containing material. In a more preferred aspect, the lignin-containing material is lignocellulose. In another preferred aspect, the inducing substrate is cellulose. In another preferred aspect, the inducing substrate is hemicellulose. In another preferred aspect, the inducing substrate is pectin. In another preferred aspect, the inducing substrate is a lipid. In another preferred aspect, the inducing substrate is phospholipid. In another preferred aspect, the inducing substrate is phytic acid. In another preferred aspect, the inducing substrate is protein. In another preferred aspect, the inducing substrate is a starch. In another preferred aspect, the inducing substrate is a medium that is low in nutrients such as amino acids, carbon, nitrogen, phosphate, or iron.

In a more preferred aspect, the protein substrate is blood, casein, egg, gelatin, gluten, milk protein, or soy protein. In another more preferred aspect, the lignin-containing material is hardwood thermomechanical pulp. In another more preferred aspect, the lignocellulose is corn stover. In another more preferred aspect, the lignocellulose is white poplar. In another more preferred aspect, the lignocellulose is rice straw. In another more preferred aspect, the lignocellulose is switch grass.

Microbial Strains. In the methods of the present invention, the microbial strain may be any microbial strain. The strain is cultured on a suitable nutrient medium with and without a substrate of interest. The strain cultured on medium without the substrate is used as a reference for identifying differences in expression of the same or similar complement of genes in the strain cultured on medium with substrate. The strain may be a wild-type, mutant, or recombinant strain.

In the methods of the present invention, the microbial strain is preferably a bacterium. In a more preferred aspect, the bacterium is a Bacillus, Pseudomonas, Streptococcus, or Streptomyces strain or E. coli.

The Bacillus strain may be any Bacillus strain. In a preferred aspect, the Bacillus strain is Bacillus alkalophilus, Bacillus amyloliquefaciens, Bacillus brevis, Bacillus cereus, Bacillus circulans, Bacillus clausii, Bacillus coagulans, Bacillus fastidiosus, Bacillus firmus, Bacillus lautus, Bacillus lentus, Bacillus licheniformis, Bacillus macerans, Bacillus megaterium, Bacillus methanolicus, Bacillus pumilus, Bacillus sphaericus, Bacillus stearothermophilus, Bacillus subtilis, or Bacillus thuringiensis. It will be understood that the term “Bacillus” also encompasses relatives of Bacillus such as Paenibacillus, Oceanobacillus, and the like.

The Pseudomonas strain may be any Pseudomonas strain. In a preferred aspect, the Pseudomonas strain is Pseudomonas acidovorans, Pseudomonas aeruginosa, Pseudomonas alcaligenes, Pseudomonas anguilliseptica, Pseudomonas abtimicrobica, Pseudomonas aurantiaca, Pseudomonas aureofaciens, Pseudomonas beijerinckii, Pseudomonas boreopolis, Pseudomonas chlororaphis, Pseudomonas citronellolis, Pseudomonas cocovenenans, Pseudomonas diminuta, Pseudomonas doudoroffii, Pseudomonas echinoides, Pseudomonas elongata, Pseudomonas fluorescens, Pseudomonas fragi, Pseudomonas halophobica, Pseudomonas huttiensis, Pseudomonas indigofera, Pseudomonas lanceolata, Pseudomonas lemoignei, Pseudomonas lundensis, Pseudomonas mendocina, Pseudomonas mephitica, Pseudomonas mucidolens, Pseudomonas oleovorans, Pseudomonas phenazinium, Pseudomonas pictorium, Pseudomonas putida, Pseudomonas resinovorans, Pseudomonas saccharophila, Pseudomonas stanieri, Pseudomonas stutzeri, Pseudomonas taetrolens, or Pseudomonas vesicularis.

The Streptococcus strain may be any Streptococcus strain. In a preferred aspect, the Streptococcus strain is a Streptococcus equisimilis cell. In another preferred aspect, the Streptococcus strain is a Streptococcus pyogenes cell. In another preferred aspect, the Streptococcus strain is a Streptococcus uberis cell. In another preferred aspect, the Streptococcus strain is a Streptococcus equi subsp. Zooepidemicus cell.

The Streptomyces strain may be any Streptomyces strain. In a preferred aspect, the Streptomyces strain is a Streptomyces achromogenes cell. In another preferred aspect, the Streptomyces strain is a Streptomyces avermitilis cell. In another preferred aspect, the Streptomyces strain is a Streptomyces coelicolor cell. In another preferred aspect, the Streptomyces strain is a Streptomyces griseus cell. In a preferred aspect, the Streptomyces strain is Streptomyces lividans. In another preferred aspect, the Streptomyces strain is Streptomyces murinus.

Microarrays. The term “an array of Bacillus licheniformis polynucleotides” is defined herein as a linear or two-dimensional array of preferably discrete elements of an array of Bacillus licheniformis polynucleotides selected from the group consisting of SEQ ID NOs: 1, 2, 3, 4, 5, 6, full-length complementary strands of SEQ ID NOs: 1, 2, 3, 4, 5, 6, and fragments of SEQ ID NOs: 1, 2, 3, 4, 5, 6 (e.g., synthetic oligonucleotides of, for example, 20-60 nucleotides), wherein each discrete element has a finite area, formed on the surface of a solid support. It is understood herein that the term “Bacillus licheniformis polynucleotides” encompasses the polynucleotides of SEQ ID NOs: 1, 2, 3, 4, 5, 6, full-length complementary strands of SEQ ID NOs: 1, 2, 3, 4, 5, 6, and fragments of SEQ ID NOs: 1, 2, 3, 4, 5, 6.

The term “microarray” is defined herein as an array of Bacillus licheniformis polynucleotide elements having a density of discrete of Bacillus licheniformis polynucleotide elements of at least about 100/cm², and preferably at least about 1000/cm². The Bacillus licheniformis polynucleotide elements in a microarray have typical dimensions, e.g., diameters, in the range of between about 10 to about 250 μm, preferably in the range of between about 10 to about 200 μm, more preferably in the range of between about 20 to about 150 μm, even more preferably in the range of between about 20 to about 100 μm, most preferably in the range of between about 50 to about 100 μm, and even most preferably in the range of between about 80 to about 100 μm, and are separated from other polynucleotide elements in the microarray by about the same distance.

Methods and instruments for forming microarrays on the surface of a solid support are well known in the art. See, for example, U.S. Pat. No. 5,807,522; U.S. Pat. No. 5,700,637; and U.S. Pat. No. 5,770,151. The instrument may be an automated device such as described in U.S. Pat. No. 5,807,522.

The term “a substrate containing an array of Bacillus licheniformis polynucleotides” is defined herein as a solid support having deposited on the surface of the support one or more (several) of a plurality of Bacillus licheniformis polynucleotides, as described herein, for use in detecting binding of labeled nucleic acids to the Bacillus licheniformis polynucleotides.

The substrate may, in one aspect, be a glass support (e.g., glass slide) having a hydrophilic or hydrophobic coating on the surface of the support, and an array of distinct random nucleic acid fragments bound to the coating, where each distinct random nucleic acid fragment is disposed at a separate, defined position.

Each microarray in the substrate preferably contains at least 10³ distinct Bacillus licheniformis in a surface area of less than about 5 or 6 cm². Each distinct Bacillus licheniformis polynucleotide (i) is disposed at a separate, defined position on the array, (ii) has a length of at least 50 bp, and (iii) is present in a defined amount between about 0.1 femtomoles and 100 nanomoles or higher if necessary.

For a hydrophilic coating, the glass slide is coated by placing a film of a polycationic polymer with a uniform thickness on the surface of the slide and drying the film to form a dried coating. The amount of polycationic polymer added should be sufficient to form at least a monolayer of polymers on the glass surface. The polymer film is bound to the surface via electrostatic binding between negative silyl-OH groups on the surface and charged cationic groups in the polymers. Such polycationic polymers include, but are not limited to, polylysine and polyarginine.

Another coating strategy employs reactive aldehydes to couple DNA to the slides (Schena et al., 1996, Proceedings of the National Academy of Science USA 93: 10614-10619; Heller at al., 1997, Proceedings of the National Academy of Science USA 94: 2150-2155).

Alternatively, the surface may have a relatively hydrophobic character, i.e., one that causes aqueous medium deposited on the surface to bead. A variety of known hydrophobic polymers, such as polystyrene, polypropylene, or polyethylene, have desirable hydrophobic properties, as do glass and a variety of lubricant or other hydrophobic films that may be applied to the support surface. A support surface is “hydrophobic” if an aqueous droplet applied to the surface does not spread out substantially beyond the area size of the applied droplet, wherein the surface acts to prevent spreading of the droplet applied to the surface by hydrophobic interaction with the droplet.

In another aspect, the substrate may be a multi-cell substrate where each cell contains a microarray of Bacillus licheniformis and preferably an identical microarray, formed on a porous surface. For example, a 96-cell array may typically have array dimensions between about 12 and 244 mm in width and 8 and 400 mm in length, with the cells in the array having width and length dimension of 1/12 and ⅛ the array width and length dimensions, respectively, i.e., between about 1 and 20 in width and 1 and 50 mm in length.

The solid support may include a water-impermeable backing such as a glass slide or rigid polymer sheet, or other non-porous material. Formed on the surface of the backing is a water-permeable film, which is formed of porous material. Such porous materials include, but are not limited to, nitrocellulose membrane nylon, polypropylene, and polyvinylidene difluoride (PVDF) polymer. The thickness of the film is preferably between about 10 and 1000 μm. The film may be applied to the backing by spraying or coating, or by applying a preformed membrane to the backing.

Alternatively, the solid support may be simply a filter composed of nitrocellulose, nylon, polypropylene, or polyvinylidene difluoride (PVDF) polymer, or, for that matter, any material suitable for use.

The film surface may be partitioned into a desirable array of cells by water-impermeable grid lines typically at a distance of about 100 to 2000 μm above the film surface. The grid lines can be formed on the surface of the film by laying down an uncured flowable resin or elastomer solution in an array grid, allowing the material to infiltrate the porous film down to the backing, and then curing the grid lines to form the cell-array substrate.

The barrier material of the grid lines may be a flowable silicone, wax-based material, thermoset material (e.g., epoxy), or any other useful material. The grid lines may be applied to the solid support using a narrow syringe, printing techniques, heat-seal stamping, or any other useful method known in the art.

Each well preferably contains a microarray of distinct Bacillus licheniformis polynucleotides. “Distinct Bacillus licheniformis polynucleotides” as applied to the polynucleotides forming a microarray is defined herein as an array member that is distinct from other array members on the basis of a different Bacillus licheniformis polynucleotide sequence or oligo sequence thereof, and/or different concentrations of the same or distinct Bacillus licheniformis polynucleotides and/or different mixtures of distinct Bacillus licheniformis polynucleotides or different-concentrations of Bacillus licheniformis polynucleotides. Thus an array of “distinct Bacillus licheniformis polynucleotides” may be an array containing, as its members, (i) distinct Bacillus licheniformis genes that may have a defined amount in each member, (ii) different, graded concentrations of a specific Bacillus licheniformis polynucleotide, and/or (iii) different-composition mixtures of two or more distinct Bacillus licheniformis polynucleotides.

It will be understood, however, that in the methods of the present invention, any type of substrate known in the art may be used.

The delivery of a known amount of a selected Bacillus licheniformis polynucleotide to a specific position on the support surface is preferably performed with a dispensing device equipped with one or more (several) tips for insuring reproducible deposition and location of the Bacillus licheniformis polynucleotides and for preparing multiple arrays. Any dispensing device known in the art may be used in the methods of the present invention. See, for example, U.S. Pat. No. 5,807,522.

For liquid-dispensing on a hydrophilic surface, the liquid will have less of a tendency to bead, and the dispensed volume will be more sensitive to the total dwell time of the dispenser tip in the immediate vicinity of the support surface.

For liquid-dispensing on a hydrophobic surface, flow of fluid from the tip onto the support surface will continue from the dispenser onto the support surface until it forms a liquid bead. At a given bead size, i.e., volume, the tendency of liquid to flow onto the surface will be balanced by the hydrophobic surface interaction of the bead with the support surface, which acts to limit the total bead area on the surface, and by the surface tension of the droplet, which tends toward a given bead curvature. At this point, a given bead volume will have formed, and continued contact of the dispenser tip with the bead, as the dispenser tip is being withdrawn, will have little or no effect on bead volume.

The desired deposition volume, i.e., bead volume, formed is preferably in the range 2 pl (picoliters) to 2 nl (nanoliters), although volumes as high as 100 nl or more may be dispensed. It will be appreciated that the selected dispensed volume will depend on (i) the “footprint” of the dispenser tip(s), i.e., the size of the area spanned by the tip(s), (ii) the hydrophobicity of the support surface, and (iii) the time of contact with and rate of withdrawal of the tip(s) from the support surface. In addition, bead size may be reduced by increasing the viscosity of the medium, effectively reducing the flow time of liquid from the dispensing device onto the support surface. The drop size may be further constrained by depositing the drop in a hydrophilic region surrounded by a hydrophobic grid pattern on the support surface.

At a given tip size, bead volume can be reduced in a controlled fashion by increasing surface hydrophobicity, reducing time of contact of the tip with the surface, increasing rate of movement of the tip away from the surface, and/or increasing the viscosity of the medium. Once these parameters are fixed, a selected deposition volume in the desired picoliter to nanoliter range can be achieved in a repeatable fashion.

After depositing a liquid droplet of a Bacillus licheniformis polynucleotide sample at one selected location on a support, the tip may be moved to a corresponding position on a second support, the Bacillus licheniformis polynucleotide sample is deposited at that position, and this process is repeated until the random nucleic acid fragment sample has been deposited at a selected position on a plurality of supports.

This deposition process may then be repeated with another random nucleic acid fragment sample at another microarray position on each of the supports.

The diameter of each Bacillus licheniformis polynucleotide region is preferably between about 20-200 μm. The spacing between each region and its closest (non-diagonal) neighbor, measured from center-to-center, is preferably in the range of about 20-400 μm. Thus, for example, an array having a center-to-center spacing of about 250 μm contains about 40 regions/cm or 1,600 regions/cm². After formation of the array, the support is treated to evaporate the liquid of the droplet forming each region, to leave a desired array of dried, relatively flat Bacillus licheniformis polynucleotide or oligo thereof regions. This drying may be done by heating or under vacuum. The DNA can also be UV-crosslinked to the polymer coating.

Nucleic Acid Probes. In the methods of the present invention, the strains are cultivated in a nutrient medium with and without a substrate using methods well known in the art for isolation of nucleic acids to be used as probes. For example, the strains may be cultivated by shake flask cultivation, small-scale or large-scale fermentation (including continuous, batch, fed-batch, or solid state fermentations) in laboratory or industrial fermentors performed in a suitable medium. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection).

The nucleic acid probes from the microbial strains cultured on medium with and without substrate may be any nucleic acid including genomic DNA, cDNA, and RNA, and may be isolated using standard methods known in the art.

The populations of isolated nucleic acid probes may be labeled with detection reporters such as colorimetric, radioactive for example, ³²P, ³³P, or ³⁵S), fluorescent reporters, or other reporters using methods known in the art (Chen et al., 1998, Genomics 51: 313-324; DeRisi et al., 1997, Science 278: 680-686; U.S. Pat. No. 5,770,367).

In a preferred aspect, the probes are labeled with fluorescent reporters. For example, the DNA probes may be labeled during reverse transcription from the respective RNA pools by incorporation of fluorophores as dye-labeled nucleotides (DeRisi et al., 1997, supra), e.g., Cy5-labeled deoxyuridine triphosphate, or the isolated cDNAs may be directly labeled with different fluorescent functional groups. Fluorescent-labeled nucleotides include, but are not limited to, fluorescein conjugated nucleotide analogs (green fluorescence), lissamine nucleotide analogs (red fluorescence). Fluorescent functional groups include, but are not limited to, Cy3 (a green fluorescent dye) and Cy5 (red fluorescent dye).

Array Hybridization. The labeled nucleic acids from the two strains cultivated with and without substrate are then added to an array of Bacillus licheniformis polynucleotides under conditions where the nucleic acid pools from the two strains hybridize to complementary sequences of the Bacillus licheniformis polynucleotides on the array. For purposes of the present invention, hybridization indicates that the labeled nucleic acids from the two strains hybridize to the Bacillus licheniformis polynucleotides under very low to very high stringency conditions.

A small volume of the labeled nucleic acids mixture is loaded onto the substrate. The solution will spread to cover the entire microarray. In the case of a multi-cell substrate, one or more (several) solutions are loaded into each cell that stop at the barrier elements.

For nucleic acid probes of at least about 100 nucleotides in length, miroarray hybridization conditions described by Eisen and Brown, 1999, Methods of Enzymology 303: 179-205, may be used. Hybridization is conducted under a cover slip at 65° C. in 3×SSC for 4-16 hours followed by post-hybridization at room temperature after removal of the cover slip in 2×SSC, 0.1% SDS by washing the array two or three times in the solution, followed by successive washes in 1×SSC for 2 minutes and 0.2×SSC wash for two or more minutes.

Conventional conditions of very low to very high stringency conditions may also be used. Very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

The carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For shorter nucleic acid probes that are less than 50 nucleotides, microarray hybridization conditions described by Kane et al., 2000, Nucleic Acids Research 28: 4552-4557, may be used. Hybridization is conducted under a supported coverslip at 42° C. for 16-18 hours at high humidity in 50% formamide, 4.1× Denhardt's solution, 4.4×SSC, and 100 μg/ml of herring sperm DNA. Arrays are washed after removal of the coverslip in 4×SSC by immersion into 1×SSC, 0.1% SDS for 10 minutes, 0.1×SSC, 0.1% SDS twice for 10 minutes, and 0.1×SSC twice for 10 minutes.

For shorter nucleic acid probes that are about 50 nucleotides to about 100 nucleotides in length, conventional stringency conditions may be used. Such stringency conditions are defined as prehybridization, hybridization, and washing post-hybridization at 5° C. to 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

The carrier material is finally washed once in 6×SSC plus 0.1% SDS for 15 minutes and twice each for 15 minutes using 6×SSC at 5° C. to 10° C. below the calculated Tₘ.

The choice of hybridization conditions will depend on the degree of homology between the Bacillus licheniformis polynucleotides and the nucleic acid probes obtained from the strain cultured with and without inducing substrate. For example, where the nucleic acid probes and the Bacillus licheniformis polynucleotides are obtained from identical strains, high stringency conditions may be most suitable. Where the strains are from a genus or species different from which the Bacillus licheniformis polynucleotides were obtained, low or medium stringency conditions may be more suitable.

In a preferred aspect, the hybridization is conducted under low stringency conditions. In a more preferred aspect, the hybridization is conducted under medium stringency conditions. In a most preferred aspect, the hybridization is conducted under high stringency conditions.

The entire solid support is then reacted with detection reagents if needed and analyzed using standard colorimetric, radioactive, or fluorescent detection means. All processing and detection steps are performed simultaneously to all of the microarrays on the solid support ensuring uniform assay conditions for all of the microarrays on the solid support.

Detection. The most common detection method is laser-induced fluorescence detection using confocal optics (Cheung et al., 1998, Nat. Genet. 18: 225-230). The array is examined under fluorescence excitation conditions such that (i) the Bacillus licheniformis polynucleotides on the array that hybridize to the first nucleic acid probes obtained from the strain cultured without inducing substrate and to the second nucleic acid probes obtained from the strain cultured with inducing substrate produce a distinct first fluorescence emission color and a distinct second fluorescence emission color, respectively, and (ii) the Bacillus licheniformis polynucleotides on the array that hybridize to substantially equal numbers of nucleic acid probes obtained from the strain cultured without inducing substrate and from the strain cultured with inducing substrate produce a distinct combined fluorescence emission color; wherein the relative expression of the genes in the strains can be determined by the observed fluorescence emission color of each spot on the array.

The fluorescence excitation conditions are based on the selection of the fluorescence reporters. For example, Cy3 and Cy5 reporters are detected with solid state lasers operating at 532 nm and 632 nm, respectively.

However, other methods of detection well known in the art may be used such as standard photometric, colorimetric, or radioactive detection means, as described earlier.

Data Analysis. The data obtained from the scanned image may then be analyzed using any of the commercially available image analysis software. The software preferably identifies array elements, subtracts backgrounds, deconvolutes multi-color images, flags or removes artifacts, verifies that controls have performed properly, and normalizes the signals (Chen et al., 1997, Journal of Biomedical Optics 2: 364-374).

Several computational methods have been described for the analysis and interpretation of microarray-based expression profiles including cluster analysis (Eisen et al., 1998, Proc. Nat. Acad. Sci. USA 95: 14863-14868), parametric ordering of genes (Spellman et al., 1998, Mol. Biol. Cell 9: 3273-3297), and supervised clustering methods based on representative hand-picked or computer-generated expression profiles (Chu et al., 1998. Science 282: 699-705). Preferred methods for evaluating the results of the microarrays employ statistical analysis to determine the significance of the differences in expression levels. In the methods of the present invention, the difference in the detected expression level is at least about 10% or greater, preferably at least about 20% or greater, more preferably at least about 50% or greater, even more preferably at least about 75% or greater; and most preferably at least about 100% or greater.

One such preferred system is the Significance Analysis of Microarrays (SAM) (Tusher et al., 2001, Proc. Natl. Acad. Sci. USA 98: 5116-5121). Statistical analysis allows the determination of significantly altered expression of levels of about 50% or even less. The PAM (or predictive analysis for microarrays) represents another approach for analyzing the results of the microarrays (Tibshirani et al., 2002, Proc. Natl. Acad. Sci. USA 99: 6567-6572).

Cluster algorithms may also be used to analyze microarray expression data. From the analysis of the expression profiles it is possible to identify co-regulated genes that perform common metabolic or biosynthetic functions. Hierarchical clustering has been employed in the analysis of microarray expression data in order to place genes into clusters based on sharing similar patterns of expression (Eisen et al., 1998, supra). This method yields a graphical display that resembles a kind of phylogenetic tree where the relatedness of the expression behavior of each gene to every other gene is depicted by branch lengths. The programs Cluster and TreeView, both written by Michael Eisen (Eisen et al., 1998 Proc. Nat. Acad. Sci. USA 95: 14863-14868) are freely available. Genespring is a commercial program available for such analysis (Silicon Genetics, Redwood City, Calif.).

Self-organizing maps (SOMs), a non-hierarchical method, have also been used to analyze microarray expression data (Tamayo et al., 1999, Proc. Natl. Acad. Sci. USA 96: 2907-2912). This method involves selecting a geometry of nodes, where the number of nodes defines the number of clusters. Then, the number of genes analyzed and the number of experimental conditions that were used to provide the expression values of these genes are subjected to an iterative process (20,000-50,000 iterations) that maps the nodes and data points into multidimensional gene expression space. After the identification of significantly regulated genes, the expression level of each gene is normalized across experiments. As a result, the expression profile of the genome is highlighted in a manner that is relatively independent of each gene's expression magnitude. Software for the “GENECLUSTER” SOM program for microarray expression analysis can be obtained from the Whitehead/MIT Center for Genome Research. SOMs can also be constructed using the GeneSpring software package.

Isolation of Genes. Probes containing genes or portions thereof identified to be induced by the present of substrate in the medium are characterized by determining the sequence of the probe. Based on the sequence, the gene can then be isolated using methods well known in the art.

The techniques used to isolate or clone a gene include isolation from genomic DNA, preparation from cDNA, or a combination thereof. The cloning of the gene from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used. The gene may be cloned from the strain of interest, or another or related organism and thus, for example, may be a species variant of the gene.

### Methods for Monitoring Differential Expression of a Plurality of Genes

The present invention also relates to methods for monitoring differential expression of a plurality of genes in a first bacterial cell relative to expression of the same genes in one or more (several) second bacterial cells, comprising:

(a) adding a mixture of detection reporter-labeled nucleic acids isolated from the bacterial cells to a substrate containing an array of Bacillus licheniformis polynucleotides selected from the group consisting of SEQ ID NOs: 1, 2, 3, 4, 5, 6, full-length complementary strands of SEQ ID NOs: 1, 2, 3, 4, 5, 6, and fragments of SEQ ID NOs: 1, 2, 3, 4, 5, 6, under conditions where the detection reporter-labeled nucleic acids hybridize to complementary sequences of the Bacillus licheniformis polynucleotides on the array, wherein the nucleic acids from the first bacterial cell and the one or more (several) second bacterial cells are labeled with a first detection reporter and one or more (several) different second detection reporters, respectively; and

(b) examining the array under conditions wherein the relative expression of the genes in the bacterial cells is determined by the observed detection signal of each spot on the array in which (i) the Bacillus licheniformis polynucleotides on the array that hybridize to the nucleic acids obtained from either the first or the one or more (several) second bacterial cells produce a distinct first detection signal or one or more (several) second detection signals, respectively, and (ii) the Bacillus licheniformis polynucleotides on the array that hybridize to the nucleic acids obtained from both the first and one or more (several) second bacterial produce a distinct combined detection signal.

The methods of the present invention may be used to monitor global expression of a plurality of genes from a Bacillus cell, discover new genes, identify possible functions of unknown open reading frames, and monitor gene copy number variation and stability. For example, the global view of changes in expression of genes may be used to provide a picture of the way in which Bacillus cells adapt to changes in culture conditions, environmental stress, or other physiological provocation. Other possibilities for monitoring global expression include spore morphogenesis, recombination, metabolic or catabolic pathway engineering.

The methods of the present invention are particularly advantageous since one spot on an array equals one gene or open reading frame because extensive follow-up characterization is unnecessary since sequence information is available, and the Bacillus licheniformis microarrays can be organized based on function of the gene products.

Bacterial Cells. In the methods of the present invention, the two or more Bacillus cells may be any Bacillus cell where one of the cells is used as a reference for identifying differences in expression of the same or similar complement of genes in the other cell(s). In one aspect, the two or more cells are the same cell. For example, they may be compared under different growth conditions, e.g., oxygen limitation, nutrition, and/or physiology. In another aspect, one or more (several) cells are mutants of the reference cell. For example, the mutant(s) may have a different phenotype. In a further aspect, the two or more cells are of different species (e.g., Bacillus clausii and Bacillus subtilis). In another further aspect, the two or more cells are of different genera. In an even further aspect, one or more (several) cells are transformants of the reference cell, wherein the one or more (several) transformants exhibit a different property. For example, the transformants may have an improved phenotype relative to the reference cell and/or one of the other transformants. The term “phenotype” is defined herein as an observable or outward characteristic of a cell determined by its genotype and modulated by its environment. Such improved phenotypes may include, but are not limited to, improved secretion or production of a protein or compound, reduced or no secretion or production of a protein or compound, improved or reduced expression of a gene, desirable morphology, an altered growth rate under desired conditions, relief of over-expression mediated growth inhibition, or tolerance to low oxygen conditions.

The Bacillus cells may be any Bacillus cells, but preferably Bacillus alkalophilus, Bacillus amyloliquefaciens, Bacillus brevis, Bacillus cereus, Bacillus circulans, Bacillus clausii, Bacillus coagulans, Bacillus fastidiosus, Bacillus firmus, Bacillus lautus, Bacillus lentus, Bacillus licheniformis, Bacillus macerans, Bacillus megaterium, Bacillus methanolicus, Bacillus pumilus, Bacillus sphaericus, Bacillus stearothermophilus, Bacillus subtilis, or Bacillus thuringiensis cells.

In a preferred aspect, the Bacillus cells are Bacillus alkalophilus cells. In another preferred aspect, the Bacillus cells are Bacillus amyloliquefaciens cells. In another preferred aspect, the Bacillus cells are Bacillus brevis cells. In another preferred aspect, the Bacillus cells are Bacillus cereus cells. In another preferred aspect, the Bacillus cells are Bacillus circulans cells. In another preferred aspect, the Bacillus cells are Bacillus clausii cells. In another preferred aspect, the Bacillus cells are Bacillus coagulans cells. In another preferred aspect, the Bacillus cells are Bacillus fastidiosus cells. In another preferred aspect, the Bacillus cells are Bacillus firmus cells. In another preferred aspect, the Bacillus cells are Bacillus lautus cells. In another preferred aspect, the Bacillus cells are Bacillus lentus cells. In another preferred aspect, the Bacillus cells are Bacillus licheniformis cells. In another preferred aspect, the Bacillus cells are Bacillus macerans cells. In another preferred aspect, the Bacillus cells are Bacillus megaterium cells. In another preferred aspect, the Bacillus cells are Bacillus methanolicus cells. In another preferred aspect, the Bacillus cells are Bacillus pumilus cells. In another preferred aspect, the Bacillus cells are Bacillus sphaericus cells. In another preferred aspect, the Bacillus cells are Bacillus stearothermophilus cells. In another preferred aspect, the Bacillus cells are Bacillus subtilis cells. In another preferred aspect, the Bacillus cells are Bacillus thuringiensis cells.

In a more preferred aspect, the Bacillus cells are Bacillus licheniformis cells. In a most preferred aspect, the Bacillus licheniformis cells are Bacillus licheniformis SJ1904 cells.

In another more preferred aspect, the Bacillus cells are Bacillus clausii cells. In another most preferred aspect, the Bacillus clausii cells are Bacillus clausii NCIB 10309 cells.

It will be understood that the term “Bacillus” also encompasses relatives of Bacillus such as Paenibacillus, Oceanobacillus, and the like.

In the methods of the present invention, the cells are cultivated in a nutrient medium suitable for growth using methods well known in the art for isolation of the nucleic acids to be used as probes. For example, the cells may be cultivated by shake flask cultivation, small-scale or large-scale fermentation (including continuous, batch, fed-batch, or solid state fermentations) in laboratory or industrial fermentors performed in a suitable medium. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection).

Nucleic Acid Probes. The nucleic acid probes from the two or more Bacillus cells may be any nucleic acid including genomic DNA, cDNA, and RNA, and may be isolated using standard methods known in the art, as described herein. The populations of isolated nucleic acid probes may be labeled with colorimetric, radioactive, fluorescent reporters, or other reporters using methods described herein.

In a preferred aspect, the probes are labeled with fluorescent reporters, e.g., Cy3 (a green fluorescent dye) and Cy5 (red fluorescent dye), as described herein.

Array Hybridization. The labeled nucleic acids from the two or more Bacillus cells are then added to a substrate containing an array of Bacillus licheniformis polynucleotides under conditions, as described herein, where the nucleic acid pools from the two or more Bacillus cells hybridize to complementary sequences of the Bacillus licheniformis polynucleotides on the array.

Detection and Data Analysis. The same methods as described herein are used for detection and data analysis.

The present invention is further described by the following examples that should not be construed as limiting the scope of the invention.

### EXAMPLES

### Example 1

### DNA Sequencing and Genome Assembly

The genome of Bacillus licheniformis SJ1904 was sequenced by a combination of the whole genome shotgun method described by Wilson, R. K. and Mardis, E. R., 1997, In Genome Analysis: A Laboratory Manual, Vol. 1, eds. Birren, B., Green, E. D., Meyers, R. M., and Roskams, J. (Cold Spring Harbor Press, Cold Spring Harbor, N.Y.), pp. 397-454, and by the highly parallel pyrosequencing method described by Margulies et al., 2005, Genome sequencing in microfabricated high-density picolitre reactors. Nature 437: 376-380.

Genomic DNA of Bacillus licheniformis SJ1904 was isolated using the following method: A single colony was used to inoculate 20 ml of LB broth (Davis, R. W., Botstein, D., and Roth, J. R. 1980, Advanced Bacterial Genetics, Cold Spring Harbor Press, Cold Spring Harbor, N.Y.) in a sterile 125 ml Erlenmeyer flask. The culture was incubated at 37° C. overnight with agitation at 240 rpm. The resulting cells were collected by centrifugation in a 45 ml screw-cap tube for 10 minutes at 6000×g, and the cell pellet was resuspended in 5 ml of Tris-glucose buffer (50 mM Tris-HCl, pH 8.0, 50 mM glucose, 10 mM EDTA). Lysozyme was added to a final concentration of 50 μg/ml and the suspension was incubated in a 37° C. water bath for 25 minutes. Next, 200 μl of 10% SDS was added and the tube was gently inverted several times. Five milliliters of a second detergent mixture (1% BRIJ®, 1% deoxycholate, 50 mM EDTA, pH 7.5) was added, and the tube was inverted several times while incubating for 20 minutes at room temperature. An equal volume of phenol:chloroform (1:1 v/v) was added and the tube was inverted gently at room temperature for 20-30 minutes. The tube was centrifuged for 20 minutes at 12,000×g, 4° C. The top aqueous layer was carefully removed with a wide-bore pipette and placed in a clean 45 ml screw-cap tube. The phenol; chloroform extraction was repeated and 1/10 volume of 3 M sodium acetate pH 5.2 was added to the aqueous layer. Two volumes of cold ethanol were carefully layered on top and the DNA was spooled from the solution onto a sterile glass rod. Spooled DNA was carefully rinsed in 70% ethanol and resuspended in a suitable amount of TE buffer (10 mM Tris-HCl, pH 8.0, 1 mM EDTA).

Plasmid libraries were constructed using randomly-sheared and Nhe I-digested genomic DNA that was enriched for 2-3 kb fragments by preparative agarose gel electrophoresis (Berka, R. M., Schneider, P., Golightly, E. J., Brown, S. H., Madden, M., Brown, K. M., Halkier, T., Mondorf, K., and Xu, F., 1997, Appl. Environ. Microbiol. 63: 3151-3157). Approximately 24,000 random clones were sequenced using dye-terminator chemistry (Applied Biosystems, Foster City, Calif.) with ABI 377, ABI 3700, and ABI 3130XL automated sequencers yielding approximately 2× coverage of the genome. For highly parallel pyrosequencing pools of random DNA fragments were generated by shearing genomic DNA and isolating single DNA molecules by limiting dilution (Margulies et al., 2005, supra). Overall sequencing coverage from this method was approximately 20×. High quality contigs were assembled from the raw pyrosequencing data using computer software described by Margulies et al. (2005).

A combination of methods was employed for gap closure including primer walking on selected clones, and PCR-amplified DNA fragments. Sequences from dye-terminator reactions were base-called using TraceTuner 2.0 (Paracel, Inc., Pasadena, Calif.) and assembled using Phrap (Gordon D., Abajian C., and Green P., 1998, Genome Res. 8: 195-202). Phrap, Crossmatch, and Consed were used for sequence finishing (Gordon D., Abajian C., and Green P., 1998, Genome Res. 8: 195-202).

### Example 2

### Identification and Annotation of Open Reading Frames (ORFs)

Protein coding regions in the assembled genome sequence data were identified using Glimmer version 3.0 (Delcher, A, L., Harmon, D., Kasif, S., White, O. and Salzberg, S. L., 1999, Nucleic Acids Res. 27, 4636-4641), and post-processed using TiCO version 2.0 (Tech M, Morgenstern B, and Meinicke P., 2006, Nucleic Acids Res. 34 (Web Server issue): W588-90). Predicted proteins were compared to the non-redundant database Uniref100 (Bairoch A, Apweiler R, Wu C H, Barker W C, Boeckmann B, Ferro S, Gasteiger E, Huang H, Lopez R, Magrane M, Martin M J, Natale D A, O'Donovan C, Redaschi N, Yeh L S. 2005. The Universal Protein Resource [UniProt]. Nucleic Acids Res. 33: D154-159) and the Bacillus subtilis genome (SubtilList) using BLASTP with an E-value threshold of 1×10⁻⁵. InterProScan version 3.3 with database release 13.0 was used to predict function (Zdobnov, E. M. and Apweiler, R., 2001, Bioinformatics 17, 847-848). The InterPro analysis included comparison to Pfam (Bateman, A., Coin, L., Durbin, R., Finn, R. D., Hollich, V., Griffiths-Jones, S., Khanna, A., Sonnhammer, E. L. et al., 2004, Nucleic Acids Res. 32, D138-D141), TIGRfam (Haft, D. J., Selengut, J. D. and White, O., 2003, Nucleic Acids Res. 31: 371-373), Interpro (Apweiler, R., Attwood, T. K., Bairock, A., Bateman, A., Birney, E., Biswas, M., Bucher, P., Cerutti, L., Corpet, F., Croning, M. D., et al., 2001, Nucleic Acids Res. 29: 37-40), signal peptide prediction using SignalP version 3.0 (Nielsen, H., Engelbrecht, J., Brunak, S., and von Heijne, G., 1997, Protein Engineering 10: 1-6), and trans-membrane domain prediction using TMHMM version 2.0 (Krogh, A., Larsson, B., von Heijne, G. and Sonnhammer, E. L. L., 2000, J. Mol. Biol. 305, 567-580). These coding sequences (CDSs) were assigned to functional categories based on the Cluster of Orthologous Groups (COG) database (Tatusov, R. L., Natale, D. A., Garkavtsev, I. V., Tatusova, T. A., Shankavarum, U. T., Rao, B. S., Kiryutin, B., Galperin, M. Y., Federova, N. D., and Koonin, E. V. 2001. The COG database: new developments in phylogenetic classification of proteins from complete genomes. Nucleic Acids Res. 29: 22-28) with manual verification as described (Tatusov, R. L., Koonin, E. V. and Lipman, D. J., 1997, Science 278: 631-637; Koonin, E. V. and Galperin, M. Y., 2002, Sequence-Evolution-Function: Computational Approaches in Comparative Genomics (Kluwer, Boston)). Transfer RNA genes were identified using tRNAscan-SE version 1.21 (Lowe, T. M. and Eddy, S. R., 1997, Nucleic Acids Res. 25: 955-964).

TABLE 1
Predicted functions

| SEQ | | | Uniref100 | |
|---|---|---|---|---|
| ID | Gene Name & Product | Uniref100 Hit Description | Accession | Hit Organism |
| 1 | ywtF Cell envelope-related | YwtF [Bacillus licheniformis | UniRef100_Q6 | Bacillus licheniformis |
| | transcriptional attenuator | DSM 13] | 5E65 | DSM 13 |
| | YwtF | | | |
| 2 | ywtE putative hydrolase | YwtE [Bacillus licheniformis | UniRef100_Q6 | Bacillus licheniformis |
| | YwtE | DSM 13] | 5E64 | DSM 13 |
| 3 | pgdS gamma-DL-glutamyl | YwtD [Bacillus licheniformis | UniRef100_Q6 | Bacillus licheniformis |
| | hydrolase | DSM 13] | 5E63 | DSM 13 |
| 4 | pgsAA poly-gamma- | YwtB [Bacillus licheniformis | UniRef100_Q6 | Bacillus licheniformis |
| | glutamate synthesis protein | DSM 13] | 5E61 | DSM 13 |
| 5 | pgsC poly-gamma- | YwtA [Bacillus licheniformis | UniRef100_Q6 | Bacillus licheniformis |
| | glutamate synthesis, | DSM 13] | 5E60 | DSM 13 |
| | Capsule biosynthesis | | | |
| | protein | | | |
| 6 | pgsB poly-gamma- | YwsC [Bacillus licheniformis | UniRef100_Q6 | Bacillus licheniformis |
| | glutamate synthesis protein | DSM 13] | 5E59 | DSM 13 |

### Deposit of Biological Material

The following biological material has been deposited under the terms of the Budapest Treaty with the American Type Culture Collection, 10801 University Blvd. Manassas, Va. 20110-2209 USA, and given the following accession number:
Deposit Accession Number Date of Deposit
B. licheniformis SJ1904 DNA ATCC PTA-7992 Nov. 8, 2006

The strain has been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

What is claimed is:

1. A mutant Bacillus host cell which comprises a disruption or deletion of a polynucleotide encoding a polypeptide selected from the group consisting of:
(a) a polypeptide comprising an amino acid sequence having at least 80% identity with the amino acid sequence of any of SEQ ID NOs: 9185, 9186, 9187, 9189, 9190, 9191; and
(b) a polypeptide encoded by a polynucleotide comprising a nucleotide sequence which hybridizes under high stringency conditions with the nucleotide sequence of any of SEQ ID NOs: 3668, 3669, 3670, 3672, 3673, 3674, or the full-length complementary strand thereof, wherein high stringency conditions are defined as prehybridization and hybridization at 42° C. in 5×SSPE, 0.3% SDS, 200 μg/ml sheared and denatured salmon sperm DNA, and 50% formamide, and washing three times each for 15 minutes using 2×SSC, 0.2% SDS at 65° C.

2. The mutant of claim 1, wherein the polypeptide comprises the amino acid sequence of any of SEQ ID NOs: 9185, 9186, 9187, 9189, 9190, 9191; preferably the polypeptide consists of the amino acid sequence of any of SEQ ID NOs: 9185, 9186, 9187, 9189, 9190, 9191.

3. The mutant of claim 1 which comprises a disruption or deletion of a polynucleotide comprising a nucleotide sequence which hybridizes under high stringency conditions with the nucleotide sequence of any of SEQ ID NOs: 4310, 4311, 4312, 4314, 4315, 4316, or the full-length complementary strand thereof, wherein high stringency conditions are defined as prehybridization and hybridization at 42° C. in 5×SSPE, 0.3% SDS, 200 μg/ml sheared and denatured salmon sperm DNA, and 50% formamide, and washing three times each for 15 minutes using 2×SSC, 0.2% SDS at 65° C.

4. The mutant of claim 1 which comprises a disruption or deletion of a polynucleotide comprising a nucleotide sequence having at least 80% identity with the nucleotide sequence of any of SEQ ID NOs: 4310, 4311, 4312, 4314, 4315, 4316; preferably the mutant comprises a disruption or deletion of a polynucleotide comprising the nucleotide sequence of any of SEQ ID NOs: 4310, 4311, 4312, 4314, 4315, 4316; most preferably the mutant comprises a disruption or deletion of a polynucleotide consisting of the nucleotide sequence of any of SEQ ID NOs: 4310, 4311, 4312, 4314, 4315, 4316.

5. The mutant of claim 1 which produces a biologically active substance homologous or heterologous to the host cell; preferably the biologically active substance is a biopolymer, more preferably a polypeptide and most preferably an enzyme.

6. The mutant of claim 5, wherein the enzyme is an oxidoreductase, transferase, hydrolase, lyase, isomerase, or ligase; most preferably the enzyme is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.

7. A method for producing a mutant Bacillus host cell, said method comprising disrupting or deleting a polynucleotide encoding a polypeptide selected from the group consisting of:
(a) a polypeptide comprising an amino acid sequence having at least 80% identity with the amino acid sequence of any of SEQ ID NOs: 9185, 9186, 9187, 9189, 9190, 9191; and
(b) a polypeptide encoded by a polynucleotide comprising a nucleotide sequence which hybridizes under high stringency conditions with the nucleotide sequence of any of SEQ ID NOs: 4310, 4311, 4312, 4314, 4315, 4316, or the full-length complementary strand thereof, wherein high stringency conditions are defined as prehybridization and hybridization at 42° C. in 5×SSPE, 0.3% SDS, 200 μg/ml sheared and denatured salmon sperm DNA, and 50% formamide, and washing three times each for 15 minutes using 2×SSC, 0.2% SDS at 65° C.

8. The method of claim 7, wherein the polypeptide comprises the amino acid sequence of any of SEQ ID NOs: 9185, 9186, 9187, 9189, 9190, 9191; preferably the polypeptide consists of the amino acid sequence of any of SEQ ID NOs: 9185, 9186, 9187, 9189, 9190, 9191.

9. The method of claim 7 comprising disrupting or deleting a polynucleotide comprising a nucleotide sequence which hybridizes under high stringency conditions with the nucleotide sequence of any of SEQ ID NOs: 4310, 4311, 4312, 4314, 4315, 4316, or the full-length complementary strand thereof, wherein high stringency conditions are defined as prehybridization and hybridization at 42° C. in 5×SSPE, 0.3% SDS, 200 μg/ml sheared and denatured salmon sperm DNA, and 50% formamide, and washing three times each for 15 minutes using 2×SSC, 0.2% SDS at 65° C.

10. The method of claim 7 comprising disrupting or deleting a polynucleotide comprising a nucleotide sequence having at least 80% identity with the nucleotide sequence of any of SEQ ID NOs: 4310, 4311, 4312, 4314, 4315, 4316; preferably comprising disrupting or deleting a polynucleotide comprising the nucleotide sequence of any of SEQ ID NOs: 4310, 4311, 4312, 4314, 4315, 4316; most preferably comprising disrupting or deleting a polynucleotide consisting of the nucleotide sequence of any of SEQ ID NOs: 4310, 4311, 4312, 4314, 4315, 4316.

11. The method of claim 9, wherein the host cell produces a biologically active substance homologous or heterologous to the host cell; preferably the biologically active substance is a biopolymer, more preferably a polypeptide and most preferably an enzyme.

12. The method of claim 9, wherein the enzyme is an oxidoreductase, transferase, hydrolase, lyase, isomerase, or ligase; most preferably the enzyme is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.

13. A method of producing a biologically active substance comprising:
(a) cultivating the mutant cell of claim 1 under conditions conducive for production of the biologically active substance; and
(b) recovering the biologically active substance.

14. The method of claim 13, wherein the biologically active substance is homologous or heterologous to the host cell; preferably the biologically active substance is a biopolymer, more preferably a polypeptide and most preferably an enzyme.

15. The method of claim 14, wherein the enzyme is an oxidoreductase, transferase, hydrolase, lyase, isomerase, or ligase; most preferably the enzyme is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.
